(19) United States
(12) Patent Application Publication
(10) Pub. No.: US 2023/0089830 A1
(43) Pub. Date: Mar. 23, 2023

(54) PHARMACEUTICAL PREPARATION WITH TRACING FUNCTION AND DELIVERY SYSTEM THEREFOR

(71) Applicant: SUZHOU YIBEN LIFE TECHNOLOGY CO., LTD, Suzhou (CN)

(72) Inventor: Yonghua DONG, Solon, OH (US)

(73) Assignee: SUZHOU YIBEN LIFE TECHNOLOGY CO., LTD, Suzhou (CN)

(21) Appl. No.: 18/049,264

(22) Filed: Oct. 24, 2022

Related U.S. Application Data
(63) Continuation of application No. PCT/CN2021/089529, filed on Apr. 25, 2021.

(30) Foreign Application Priority Data
Apr. 24, 2020 (CN) 202010335276.0

(51) Int. Cl. A61B 8/08; A61B 6/00; A61M 5/00; A61M 5/142

(52) U.S. Cl. CPC A61B 8/481; A61B 6/481; A61M 5/007; A61M 5/142

(57) ABSTRACT

Disclosed are a pharmaceutical preparation with a tracing function, and a delivery system therefor. The pharmaceutical preparation comprises a first drug and a second drug in a liquid or gaseous state, wherein the first drug and the second drug are each divided into multiple sections, which are arranged in series at intervals in a conduit. One of the first drug and the second drug is a tracer drug that can be developed in a medical imaging device in the human body. The first drug and the second drug are immiscible and satisfy compatibility requirements. The pharmaceutical preparation can comprise an aerobic contrast agent, an aerobic embolic agent and an aerobic perfusion agent. Same can be applied to a variety of contrast techniques by means of a one-step simple operation, saving the dosage of a drug while maintaining a high concentration of the drug.

### BACKGROUND

### Technical Field

The present disclosure relates to a pharmaceutical preparation with a tracing function, and further relates to a delivery system for the pharmaceutical preparation, belonging to the technical field of medical instruments.

### Related Art

During clinical practice of interventional surgery, a doctor always mixes a nonionic iodine contrast agent with absolute alcohol, such as ioversol and iodixanol to realize the monitorability of an embolic agent. Such way dilutes the concentration of the absolute alcohol, resulting in a non-ideal embolization effect. In addition, for a liquid contrast agent, there is another problem: in clinical practice, it is needed to input the contrast agent first, then cleaning is performed, and finally the embolic agent is input. During inputting the contrast agent, it is needed to make a conduit full of the contrast agent; and during inputting the embolic agent, it is needed to make the conduit full of the embolic agent or an embolic agent solution, consequently, the use amounts of the contrast agent and the embolic agent are both relatively large, the practice is complicated, and excessive metabolic burden is easily brought to the human body.

In addition, some doctors use gaseous contrast agents under certain conditions. However, in case of using the gaseous contrast agents such as carbon dioxide, it is needed to inject a large amount of gas to empty blood in the blood vessels to realize low-density contrast so as to display blood vessel images. Although carbon dioxide can be quickly absorbed by human blood, tissue hypoxia and ischemia may be caused to a certain extent. Therefore, carbon dioxide angiography can only be performed in arteries in the region below the diaphragm muscle in clinic, and cannot be performed on heart and brain parts and organs sensitive to ischemia or hypoxia.

How to solve the problems in the prior art is still a research hotspot for those skilled in the art.

### SUMMARY

The present disclosure provides a pharmaceutical preparation with a tracing function.

The present disclosure further provides a delivery system for the pharmaceutical preparation.

To achieve the objectives, the present disclosure adopts the following technical solutions.

In one aspect, an embodiment of the present disclosure provides a pharmaceutical preparation with a tracing function, the pharmaceutical preparation includes a conduit containing a tracer drug, and a conduit head, wherein

a first drug and a second drug in a liquid or gaseous state are arranged in the conduit; the first drug and the second drug are each divided into multiple sections, which are arranged in series at intervals in the conduit; one of the first drug and the second drug is the tracer drug which can be developed in a medical imaging device in the human body; and the first drug and the second drug are immiscible, insoluble or slightly soluble, and satisfy the acceptable treatment compatibility requirements in the art.

Preferably, the first drug is a contrast agent; and the second drug is a gaseous separant.

Preferably, the first drugs in the liquid state or the second drugs in the liquid state are at two ends of the conduit.

Preferably, multiple sections of third drug in a liquid or gaseous state are further arranged in the conduit, and the third drug is arranged between the first drug and the second drug, and

the third drug is immiscible with the first drug and the second drug and satisfies the acceptable compatibility requirements in the art; and the third drug and the second drug satisfy the compatibility requirements.

Preferably, the first drug which is the tracer drug is a liquid contrast agent and positioned at the two ends of the conduit;

the second drug is a gaseous separant, and the first drug is positioned on the two sides of each section of the second drug;

the third drug is an embolic agent or a perfusion agent, and the second drug is positioned on the two sides of each section of the third drug; and

arranging the first drug, the third drug, the second drug and the third drug in the conduit from the end is served as a unit and is repeatedly arranged until the first drug is positioned at the other end of the conduit.

Preferably, the first drug is an anhydrous iodine contrast agent, the second drug is carbon dioxide, and the third drug is alcohol.

In a second aspect, the embodiment of the present disclosure provides an aerobic contrast agent, and the aerobic contrast agent includes a conduit and a conduit head, where oxygen and a liquid contrast agent are arranged in the conduit; and the oxygen and the contrast agent are each divided into multiple sections which are arranged in series at intervals in the conduit; and

the oxygen and the liquid contrast agent are immiscible, insoluble or slightly soluble, and satisfy the acceptable treatment compatibility requirements in the art.

In a third aspect, the embodiment of the present disclosure provides an aerobic embolic agent, and the aerobic embolic agent includes a conduit and a conduit head, where oxygen, a liquid contrast agent and the liquid embolic agent are arranged in the conduit; the oxygen, the contrast agent and the embolic agent are each divided into multiple sections; and the contrast agent and the embolic agent are arranged in series at intervals in the conduit through the oxygen; and

the oxygen, the liquid contrast agent and the liquid embolic agent are immiscible, insoluble or slightly soluble, and satisfy the acceptable treatment compatibility requirements in the art.

In a fourth aspect, the embodiment of the present disclosure provides an aerobic perfusion agent, and the aerobic perfusion agent includes a conduit and a conduit head, where oxygen, a liquid contrast agent and the liquid perfusion agent are arranged in the conduit; the oxygen, the liquid contrast agent and the liquid perfusion agent are each divided into multiple sections; and the contrast agent and the embolic agent are arranged in series at intervals in the conduit through the oxygen; and

the oxygen, the liquid contrast agent and the liquid perfusion agent are immiscible, insoluble or slightly soluble, and satisfy the acceptable treatment compatibility requirements in the art.

In a fifth aspect, the embodiment of the present disclosure provides a delivery system for a pharmaceutical preparation with a tracing function, the delivery system includes an injection pump, a conduit, a sheath tube holder and a puncture needle which are sequentially connected, where the conduit is the abovementioned conduit.

The pharmaceutical preparation with a tracing function provided by the embodiment of the present disclosure includes the aerobic contrast agent, the aerobic embolic agent and the aerobic perfusion agent. The pharmaceutical preparation can be applied to a variety of contrast techniques by means of a one-step simple operation, achieving clear angiography under a condition that the concentration of the embolic agent is not reduced, and saving the dosage of a drug while maintaining a high concentration of the drug. Moreover, the pharmaceutical preparation is injected into oxygen together with a drug during interventional surgery, which can increase cell activity to improve drug efficacy, thereby achieving an inhibitory effect on tumor cells; and same can also improve the flexibility of drug compatibility, thereby realizing accurate hemodynamic analysis.

### BRIEF DESCRIPTION OF THE DRAWINGS

FIG. 1 is a schematic diagram of a delivery system for a gas-liquid preparation provided by an embodiment of the present disclosure;

FIG. 2 is a schematic structure diagram of a gas-liquid preparation in an Embodiment I of the present disclosure;

FIG. 3 is a schematic structure diagram of a gas-liquid preparation in an Embodiment II of the present disclosure;

FIG. 4A is a display effect diagram of a gas-liquid preparation under X rays in an Embodiment I of the present disclosure;

FIG. 4B is a display effect diagram of a gas-liquid preparation under X rays in FIG. 4A;

FIG. 4C is a display effect diagram of a gas-liquid preparation under X rays in FIG. 4A;

FIG. 5 is a schematic structure diagram of a gas-liquid preparation in an Embodiment V of the present disclosure;

FIG. 6A is a diagram showing matching of a gas-liquid preparation conduit head end joint and a gas-liquid preparation conduit tail end joint in an Embodiment V of the present disclosure;

FIG. 6B is a schematic diagram of a conduit state in an Embodiment V of the present disclosure;

FIG. 7A is an X-ray shot picture of PE conduits under conduit voltage of 49.90 kV;

FIG. 7B is an X-ray shot picture of PE conduits under conduit voltage of 80.90 kV;

FIG. 7C is an X-ray shot picture of PE conduits under conduit voltage of 89.80 kV;

FIG. 8A is a continuous exposure acquisition diagram of injecting an An'erdian-sodium chloride mixed solution into a 10-1 PE conduit;

FIG. 8B is a continuous exposure acquisition diagram of a 10-2 PE conduit simulating conduit advancing under a condition of adding an abdominal model;

FIG. 9A is a gross observation diagram of taking out both kidneys after left renal artery embolization on an experimental rabbit 1;

FIG. 9B is a gross observation diagram of an embolized kidney (left kidney) specimen after soaking in a formalin solution for 12 h;

FIG. 9C is an observation diagram of a coronal section of left kidney;

FIG. 10A is a gross observation diagram of taking out both kidneys after both renal artery embolization of an experimental rabbit 2;

FIG. 10B is a gross observation diagram of a left kidney specimen after soaking in formalin for 12 h;

FIG. 10C is an observation diagram of a coronal section of left kidney;

FIG. 10D is a gross observation diagram of a right kidney specimen after soaking in formalin for 12 h;

FIG. 11A is an edema degeneration phenomenon observed from tubular epithelial cells of left kidney;

FIG. 11B is an elastic fiber breakage phenomenon observed from extremely individual arterial wall of left kidney;

FIG. 12A is a diagram of a cortical infarction region in right kidney;

FIG. 12B is a diagram of edema degeneration of tubular epithelial cells of left kidney;

FIG. 13A is a sample picture of a gas-liquid series embolic agent provided by an embodiment of the present disclosure, and the sample includes about 6 μl of carbon dioxide and about 15 μl of 75% alcohol;

FIG. 13B is a sample picture of a gas-liquid series embolic agent provided by an embodiment of the present disclosure, and the sample includes about 60 μl of carbon dioxide and about 15 μl of 75% alcohol;

FIG. 14A is a sample picture of a gas-liquid series embolic agent provided by an embodiment of the present disclosure, and the sample includes about 10 μl of carbon dioxide and about 15 μl of lipiodol;

FIG. 14B is a sample picture of a gas-liquid series embolic agent provided by an embodiment of the present disclosure, and the sample includes about 70 μl of carbon dioxide and about 15 μl of lipiodol; and

FIGS. 15A-15G are various angiography images in an animal experiment of the present disclosure.

### DETAILED DESCRIPTION

The technical content of the present disclosure is described in detail below with reference to the accompanying drawings and specific embodiments.

A “gas-liquid preparation” described in an embodiment of the present disclosure includes a preparation formed by alternating gas and liquid, and includes a preparation formed by alternating first liquid and second liquid (the two are immiscible). In order to make description simple, in case of particularly emphasizing a gas-liquid-gas structure, the “gas-liquid preparation” only includes the preparation formed by alternating the gas and the liquid, otherwise, the “gas-liquid preparation” includes the abovementioned two conditions.

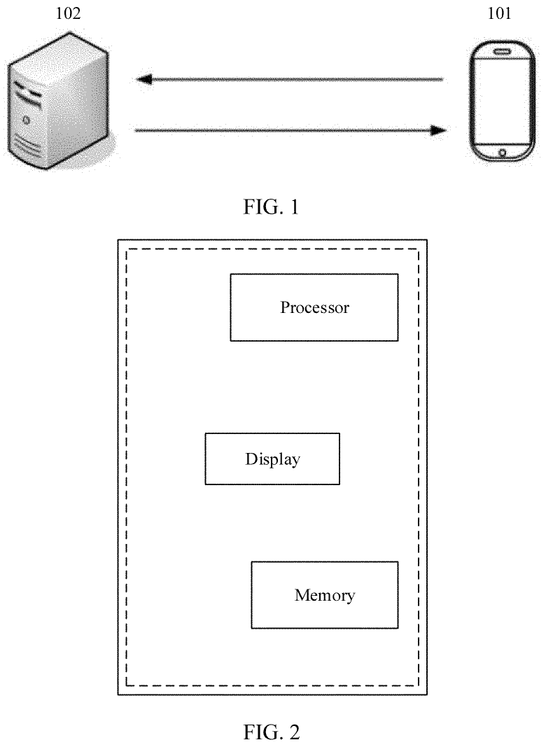

As shown in FIG. 1 and FIG. 2, a delivery system for a pharmaceutical preparation provided by the embodiment of the present disclosure includes an injection pump 100, a conduit 200, a sheath tube holder 400 and a vascular sheath 500. The injection pump 100 is connected with a tail end (the end close to the injection pump) of the conduit 200, and the other end (a far end, close to a patient) of the conduit 200 is connected with the vascular sheath 500 through the sheath tube holder 400. One end of the vascular sheath 500 is inserted into the artery blood vessel (not shown in the drawings) or human tissue (such as tumor tissue).

The injection pump 100 can be of a conventional model such as a German BRAUN micro-injection pump Perfusor Space or a double-channel micro-injection pump (WZS-50F6) produced by Zhejiang Smiths Medical Instrument Co., Ltd. according to the needs of interventional surgery, thereby realizing injection at multiple rates and multiple capacities. It is understood by those of ordinary skill in the art that manual injection is available in case of no injection pump 100.

The conduit 200 is connected with the sheath tube holder 400 through a Luer taper. The Luer taper conforms to the regulations of Chinese standard GB/T 1962.2-2001 or the international standard ISO 594-2-1998 on Injector, Needle and 6% (Luer) Conical Taper of Other Medical Instruments Part 2: Locking Taper, and can be used for quick connection of available medical instruments.

The sheath tube holder 400 conforms to the requirements of industrial standards YY0450.1-2003 and YY0258.2-2004, and is connected with a side branch tube 300.

The conduit 200 includes a conduit body 1 and a conduit head 2 (Luer taper). The conduit body 1 is of a slender tubular structure, and two ends of the conduit body are closed by the conduit head 2. The conduit body 1 is made of plastic, resin or glass or other materials, preferably high-performance polyolefin thermoplastic elastomer (TPE) such as novel MT-12051 type TPE materials produced by Polymax TPE Company.

The normal average lumen diameter of artery blood vessel is as follows: the diameter of elastic artery is about 15 mm, the diameter of muscle artery is about 6 mm, the diameter of arteriolar is about 37 μm, and the diameter of capillary vessel is about 9 μm. The outer diameter and the inner diameter of the conduit for the gas-liquid preparation provided by the embodiment of the present disclosure have various specifications, the inner diameter range includes but is not limited to 0.2-15 mm, preferably 0.5-8 mm, and the outer diameter of the conduit can be smaller than or equal to the inner diameter of the artery blood vessel in case of selecting the appropriate specifications. If the outer diameter of the conduit is reduced, the inner diameter will be correspondingly reduced, and thus the flow resistance of gas or liquid in the conduit body 1 will be increased. The small inner diameter will make the gas or the liquid difficult to flow in case of not applying pressure on the gas or liquid in the conduit body 1, so that the gas or the liquid will not move relatively (not be mutually suspended) even if vibration is applied from the outside, and the small inner diameter is particularly applicable to the liquid-liquid preparation which takes liquid as a separant, such as first liquid-second liquid-first liquid-second liquid. The larger inner diameter, such as the inner diameter of 2 mm or above, is applicable to taking gas as a separant for liquid. However, the pharmaceutical preparation provided by the embodiment of the present disclosure can also be used for treating hemangioma, liver cancer, brain tumor and the like, and is not limited to artery.

The conduit head 2 includes a male head 2A and a female head 2B which are respectively positioned at two ends of the conduit body 1 and used for closing/sealing liquid or gas in the conduit body 1. The conduit head 2 is a standard Luer taper. Because one end of the conduit body is the male head 2A and the other end is the female head 2B, two conduit bodies 1 can be connected by butting the male head of one conduit body with the female head of the other conduit body, thereby realizing the connection of multiple conduit bodies 1 and increasing the drug dosage (drugs in the multiple conduit bodies 1 can be continuously supplied). In addition, because the standard Luer taper (national standard GB/T 1962.2-2001) is adopted, the conduit body 1 can be conveniently connected to various conventional injectors or other medical instruments through the Luer taper, and thus the gaseous or liquid drug in the conduit body 1 can be input into the human body or the animal body through a conventional injector and the like. Before use, the male and female Luer tapers at two ends of the conduit 200 can be connected in the storage and transportation stage, thus increasing the sealing property of the conduit in the storage and transportation process, and reducing the size of the conduit package.

The drug in the conduit body 1 exists in a form of gas or liquid (including suspension). The drug in the conduit body 1 can include different types of drugs, such as an embolic agent, a perfusion agent, a chemical ablation agent, a developer and an anesthetic.

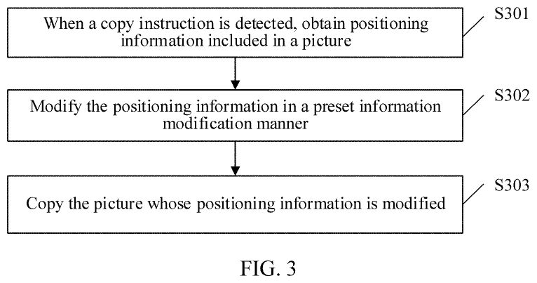

The drug in the conduit body 1 has multiple combination forms, such as a staggered or spaced form of liquid (contrast agent)-gas spacer (oxygen)-liquid (contrast agent)-gas spacer (oxygen) (see FIG. 2), a staggered or spaced form of first liquid (contrast agent)-second liquid (embolic agent)-first liquid (contrast agent)-second liquid (embolic agent), or a staggered or spaced form of first liquid (contrast agent)-gas spacer (oxygen)-second liquid (embolic agent)-gas spacer (oxygen)-first liquid (contrast agent)-gas spacer (oxygen)-second liquid (embolic agent) (see FIG. 3). In other words, the drug can be of the staggered form of liquid and gas or the staggered form of liquid and liquid.

Preferably, the liquid (such as contrast agent) is arranged at two ends (head and tail) of the conduit body 1, thus, on one hand, an image of liquid (contrast agent) at two ends can be conveniently seen for angiography imaging, and the position of the gas-liquid preparation in the whole conduit body 1 can be positioned; and on the other hand, the gas tightness can be improved, and gas leakage is prevented.

### Embodiment I

The Embodiment I of the present disclosure provided an aerobic contrast agent. In the Embodiment I of the present disclosure, the total capacity of the conduit body 1 was 10 mL, and the length was 1 m. The capacity of the conduit body 1 depended on the drug dosage and the medication speed of the interventional surgery, and could be set to be lengths of different specifications of 400 mm, 600 mm and 800 mm and corresponding inner diameters. If the capacity for the interventional surgery exceeded the total capacity (like 10 mL) of one conduit body, multiple gas-liquid preparations could be connected (a male Luer taper and a female Luer taper of two adjacent gas-liquid preparations were connected). 2 drugs, namely the contrast agent (a first drug 11) and the separant (a second drug 12) were contained in the conduit body 1 and were used for angiography in the interventional therapy. As shown in FIG. 2, the first drug 11 was a liquid contrast agent that was ioversol; and the second drug 12 was a gaseous separant that was oxygen. The oxygen 12 was used for separating the sections of the contrast agent 11 (a staggered form of liquid-gas-liquid-gas), the total amount of gas in the conduit body 1 could not be greater than 0.8 mL so as to avoid discomfort of the human body, and the amount of gas in each section could not be greater than 0.1 mL so as to avoid embolization. The compatibility of the drugs in the conduit would satisfy the reasonable design in the aspects of physics, chemistry and curative effect, and would conform to the regulations of pharmaceutics, especially incompatibility in the aspects of physics and chemistry. For example, in case of first liquid and second liquid in the conduit, the first liquid and the second liquid might be separated out and precipitated due to the change of solubility. Therefore, the liquid was prevented from being turbid or precipitated caused by the mixing through the separant (like gas). For another example, if carbon dioxide was used as the separant, the PH value of the liquid adjacent to the carbon dioxide would be changed, consequently, some strongly alkaline liquid drugs might be separated out and precipitated due to the change of the PH value, and thus, it was needed to use oxygen as the separant or use the contrast agent as the separant for such liquid drugs.

The first drug and the second drug were each divided into multiple sections, and the sections of the first drug were equal in length, and the length was marked as L1; and the sections of the second drug were equal in length, and the length was marked as L2. It was understood by those of ordinary skill in the art that the sections might be not equal in length, and it was not limited to uniform division. The section length L1 of the first drug (contrast agent) was equal to or larger than the section length L2 of the second drug (separant). It was assumed that if L1=L2 (the sections of the first drug and the second drug were equal in length), the length of the conduit body 1 was that L1=L2=L/(2N+1), where L was the length of the conduit 1, moreover, the number of the sections of the first drug (contrast agent) was 2N*L/(2N+1), and the number of the sections of the second drug was N*L/(2N+1).

The design of the length L1 or L2 of each section needed to satisfy 1) drug dosage control of each section, and 2) control of the total amount of each drug in the whole conduit body 1. The drug dosage control of each section was affected by the flowing property of the drug in the conduit body 1. If the flowing property was low, the drug dosage of each section was small, that was, the length of the drug in each section was short; and if the flowing property was high, the drug dosage of each section was large. The total amount control of each drug in the conduit body 1 was affected by the safe dosage for human, which was well known by doctors.

In this embodiment, the length L2 of each section of the second drug that was the separant oxygen was smaller than the length L1 of each section of the first drug that was the contrast agent. On one hand, this was because the section length of oxygen was too long, which might cause the flowing of the contrast agent (the contrast agent could not be pushed); and on the other hand, bubbles formed after oxygen entered blood could not be too large, otherwise, the human body might feel uncomfortable. That was, the first drug (contrast agent) and the second drug (separant) were immiscible, insoluble or slightly soluble, and satisfied the acceptable treatment compatibility requirements in the art.

During angiography, the conduit body 1 was pressurized by the injection pump 100 (or manual injection instead of the injection pump), so the first drug (contrast agent) 11 at the head end of the conduit body 1 first entered the blood vessel; and then the first drug (contrast agent) 11 at the tail end of the conduit body 1 entered the blood vessel. Since the first drug at the head end and the first drug at the tail end were both contrast agents, imaging could be performed under X-rays, and the position of liquid 120 at the head end and the position of liquid 121 at the tail end could be seen on an image picture.

### Embodiment II

As shown in FIG. 3, in the Embodiment II of the present disclosure, a first drug 11A was a liquid contrast agent (ioversol) and was positioned at two ends of the conduit 200; and a second drug 12A was a gaseous separant (carbon dioxide), and each section of the first drug was positioned at two sides of one section of the second drug. A third drug 13 was an embolic agent or a perfusion agent (75% alcohol in this embodiment), and each section of the third drug 13 was between two sections of the second drug 12A. In the conduit 200, the drugs were arranged in series from the head of the conduit in a sequence of liquid contrast agent (ioversol)-gaseous spacer (carbon dioxide)-embolic agent or perfusion agent (alcohol)-gaseous spacer (carbon dioxide), the arrangement serving as a unit was repeated in the conduit and the liquid contrast agent added at the tail of the conduit served as a final drug. The first drug (contrast agent), the second drug (separant) and the third drug (embolic agent or perfusion agent) were immiscible, insoluble or slightly soluble, and satisfied the acceptable treatment compatibility requirements in the art.

As shown in FIG. 3, the volume ratio (length ratio) of three drugs in the conduit 1 was: ioversol N+1:carbon dioxide 2N:75% alcohol N, where L1=L2=L3=L/(4N+1), L was the length of the conduit 1, and L1, L2 and L3 were the length of each section of the first drug, the length of each section of the second drug and the length of each section of the third drug respectively. It was understood by those of ordinary skill in the art that L1, L2 and L3 might also change according to the drug dosage and were not necessarily equal. The assumption that the three lengths were equal was only a simplified description for making understanding easy, but would not cause any limitation to the present disclosure.

Similar to the Embodiment I, the gas-liquid preparation of the Embodiment II of the present disclosure would reduce the dosage of the contrast agent and the embolic agent, as it was not needed to make the whole target blood vessel full of the embolic agent (it was assumed that the dosage required for fully filling was V_{target blood vessel}), and the embolic agent of N*V_{target blood vessel}/(4N+¹) could fully fill the target blood vessel together with the contrast agent and the separant.

Moreover, the Embodiment II of the present disclosure would trace the position of the embolic agent under X rays, which was mainly realized through the liquid contrast agent and the carbon dioxide contrast agent. Because carbon dioxide obstructed the mixing of the liquid contrast agent and the embolic agent, the relative concentration of the embolic agent was not influenced by the contrast agents (the embolic agent did not contact blood and could not be diluted by the blood), and thus the optimal embolization performance of the embolic agent was realized. Meanwhile, the pharmaceutical preparation of this embodiment also prolonged the contact time of the embolic agent and the target blood vessel. Because the embolic agent (the third drug 13) was between two second drugs (gaseous separant), the flow rate of the embolic agent was less than that of the embolic agent (without gas) injected in the conventional interventional therapy through gas separation, and the contact time of the embolic agent and cells in the blood vessel was correspondingly prolonged. Moreover, the osmotic pressure of tissues in tumor was high, and the embolic agent was not diluted by the blood and kept high concentration (the osmotic pressure was higher than that of the conventional preparation), so the embolic agent easily permeated into the microvessels of the tumor and diffused to denature and necrotize tumor cells.

The Embodiment II of the present disclosure could be suitable for photoacoustic angiography (B-ultrasound) and X-ray angiography (CT), and could also be conveniently used for performing hemodynamic monitoring.

Since the second drug that was carbon dioxide was gas without physiological hazard, and the solubility of the carbon dioxide in blood was 2.3 times that of oxygen, aeroembolism was not easy to occur. The carbon dioxide was also a gaseous negative contrast agent, which could be used for angiography; and after entering the blood, the carbon dioxide could be dissolved in the blood, and was discharged from the lung when reaching the lung circulation. Therefore, the carbon dioxide was a contrast agent which did not increase the circulation burden and did not cause allergic reactions. However, since the carbon dioxide was inconvenient to store, a carbon dioxide machine was required for clinical preparation during angiography; in clinical practice, if the carbon dioxide was filled into the embolic agent, a microbubble structure could be realized, but the tracing property of the microbubble structure was poor, the uniformity could not be controlled, and the microbubble structure was easy to eliminate; and the above problems limited the wide clinical application to a certain extent. However, since the carbon dioxide had high water solubility but was insoluble in an iodine solution, the carbon dioxide could be compatible with an anhydrous iodine contrast agent, and the carbon dioxide and alcohol were immiscible, so the effect of well isolating the contrast agent and the embolic agent could be achieved, the drug properties of the contrast agent and the embolic agent were not affected, and the flexibility of drug compatibility was improved.

### Embodiment III

This embodiment provided an aerobic embolic agent, and the aerobic embolic agent included a first drug 11 which was a liquid contrast agent (ioversol) at two ends of the conduit, a second drug 12 which was a gaseous separant (oxygen) and of which each section was on two sides of one section of the second drug, and a third drug which was an embolic agent such as 75% alcohol or lipiodol, an arterial chemotherapy embolic agent, a radiotherapy embolic agent and a microsphere suspension. The structure of the pharmaceutical preparation was similar to that of the Embodiment II, so no more description was made herein. The aerobic embolic agent in this embodiment included the conduit and the conduit head, and oxygen, a liquid contrast agent and a liquid embolic agent were arranged in the conduit; the oxygen, the contrast agent and the embolic agent were each divided into multiple sections; and the contrast agent and the embolic agent were arranged in series at intervals in the conduit through the oxygen. The oxygen, the contrast agent and the embolic agent were immiscible, insoluble or slightly soluble, and satisfied the acceptable treatment compatibility requirements in the art.

### Embodiment IV

This embodiment provided an aerobic perfusion agent, and the aerobic perfusion agent included the first drug 11 which was the liquid contrast agent (ioversol) at two ends of the conduit, the second drug 12 which was the gaseous separant (oxygen) and of which each section was on two sides of one section of the second drug, and the third drug which was the perfusion agent such as the perfusion drug used in TAI, TAE and TACE, a chemotherapeutic drug for arterial perfusion, a radioactive particle suspension and a microsphere suspension.

The aerobic perfusion agent in this embodiment included the conduit and the conduit head, and oxygen, a liquid contrast agent and a liquid perfusion agent were arranged in the conduit; the oxygen, the contrast agent and the perfusion agent were each divided into multiple sections; and the contrast agent and an embolic agent were arranged in series at intervals in the conduit through the oxygen. The oxygen, the contrast agent and the perfusion agent were immiscible, insoluble or slightly soluble, and satisfied the acceptable treatment compatibility requirements in the art.

The structure of the pharmaceutical preparation was similar to that of the Embodiment II, so no more description was made herein.

The technical solution and technical advantages of the present disclosure were introduced by combining different embodiments. The separant used in the present disclosure could be oxygen or carbon dioxide, and could also be super oxygen (O₃) and other gases safe to the human body. The contrast agent used in the present disclosure included an X-ray contrast agent, which could be an ionic contrast agent, and could also be a non-ionic contrast agent; the contrast agent further included an MRI contrast agent, which could be a macromolecular paramagnetic developer and a nanostructure developer; and the contrast agent further included an ultrasonic developer, such as a liquid fluorocarbon nano-emulsion.

### Embodiment V

In the Embodiment V of the present disclosure, the first drug was the embolic agent (75% alcohol); the second drug was the gaseous separant (carbon dioxide); and in order to prevent gas loss, the drugs at two ends of the conduit 200 were liquid embolic agents. In this embodiment, arranging as alcohol-embolic agent from the head of the conduit was treated as a unit and repeatedly circularly performed; and alcohol was added at the tail of the conduit 200. In this embodiment, the ratio of the length of the first drug (75% alcohol) grain to the length of the second drug (carbon dioxide) was 1; and since alcohol had certain volatility, in order to ensure the embolization effect of alcohol, the length of the alcohol grain could be properly prolonged, for example, the length ratio of alcohol to carbon dioxide could be 2:1. In this embodiment, the total length of the conduit was 100 cm, and the inner diameter was 2.0 mm. The ratio of the length to the inner diameter of the conduit was also particularly important; since the gaseous drug and the liquid drug were arranged at intervals in the conduit, if the total length of the conduit was too short, the drug containing amount was limited; and if the inner diameter was increased in order to increase the drug containing amount, the loss of the drug in the transportation and storage process could be increased especially under the condition that the embolic agent was a volatile liquid drug. If the length of the conduit was too long and the inner diameter was too small, the drug injecting resistance could be increased; and if the inner diameter was too large, the volume of the conduit and the drug dosage could be increased, thus causing drug loss and waste. The optimal ratio of the length to the inner diameter of the conduit could make drug application convenient and effective in surgery under the condition of acceptable drug loss.

### Embodiment VI

In the abovementioned Embodiment V, the conduit 200 was pre-filled with filler and stored for standby application. That is, the liquid drug was injected into the conduit 200 in a pharmaceutical factory, the conduit heads 2 were closed, and then storage, transportation and the like were conducted; and during surgery, the doctor opened one or two of the conduit heads 2 and connected to an operation instrument, and then injected the filler in the conduit 200 into a living body. This mode was called as a pre-filling mode. In this embodiment, the near end of the conduit 200 was directly connected with an injection device while the far end was connected with a needle. That is, the liquid drug was injected into the conduit 200 while the conduit was connected to the needle through the conduit head 2 at the far end during surgery, so that liquid or gas in the conduit 200 entered the living body. This mode was called as a field-filling mode.

As shown in FIGS. 13A-14B, part of experimental data was provided, which were obtained on the basis of the field-filling mode. However, it was understood by those of ordinary skill in the art that the same experimental data could be obtained in the pre-filling mode.

| | Preparation | | | | Carbon | | | | | | |
|---|---|---|---|---|---|---|---|---|---|---|---|
| | conditions | | | | dioxide | | | | | | |
| | (minimum/ | | | | volume of | | | | | | |
| | maximum gas | | Conduit | Inner | single | Gas | Liquid | | Liquid | Injecting | |
| | volume of single | Conduit | length | diameter | section | pressure | temperature | | volume | speed | Preparation |
| No. | bubble) | number | (mm) | (mm) | (μl) | (kpa) | (° C.) | Liquid | (μ1) | (μ1/s) | time |
| 1 | Different liquid | 1 | 1000 | 2 | 6 | 15 | Room | 75% | 15 | 15 | 2021 Apr. 7 |
| | (water phase, oil | | | | | | temperature | alcohol | | | |
| | phase) | 2 | 1000 | 2 | 60 | 15 | Room | 75% | 15 | 15 | 2021 Apr. 7 |
| | | | | | | | temperature | alcohol | | | |
| | | 3 | 1000 | 2 | 10 | 15 | Room | Lipiodol | 15 | 15 | 2021 Apr. 7 |
| | | | | | | | temperature | | | | |
| | | 4 | 1000 | 2 | 70 | 15 | Room | Lipiodol | 15 | 15 | 2021 Apr. 7 |
| | | | | | | | temperature | | | | |
| 2 | Different | 5 | 1000 | 2 | 7 | 15 | 6 | 75% | 15 | 15 | 2021 Apr. 7 |
| | temperature | | | | | | | alcohol | | | |
| | | 6 | 1000 | 2 | 70 | 15 | 6 | 75% | 15 | 15 | 2021 Apr. 7 |
| | | | | | | | | alcohol | | | |
| | | 7 | 1000 | 2 | 8 | 15 | 45 | 75% | 15 | 15 | 2021 Apr. 7 |
| | | | | | | | | alcohol | | | |
| | | 8 | 1000 | 2 | 70 | 15 | 45 | 75% | 15 | 15 | 2021 Apr. 7 |
| | | | | | | | | alcohol | | | |
| 3 | Different conduit | 9 | 1000 | 1 | 5 | 15 | Room | 75% | 15 | 15 | 2021 Apr. 8 |
| | inner diameter | | | | | | temperature | alcohol | | | |
| | | 10 | 1000 | 1 | 30 | 15 | Room | 75% | 15 | 15 | 2021 Apr. 8 |
| | | | | | | | temperature | alcohol | | | |
| 4 | Different pressure | 11 | 1000 | 2 | 15 | 5 | Room | 75% | 15 | 15 | 2021 Apr. 8 |
| | | | | | | | temperature | alcohol | | | |
| | | 12 | 1000 | 2 | 60 | 5 | Room | 75% | 15 | 15 | 2021 Apr. 8 |
| | | | | | | | temperature | alcohol | | | |
| | | 13 | 1000 | 2 | 3 | 40 | Room | 75% | 15 | 15 | 2021 Apr. 8 |
| | | | | | | | temperature | alcohol | | | |
| | | 14 | 1000 | 2 | 60 | 40 | Room | 75% | 15 | 15 | 2021 Apr. 8 |
| | | | | | | | temperature | alcohol | | | |

The gas-liquid pharmaceutical preparation with different compatibility provided by the embodiment of the present disclosure could be adopted to respectively obtain the following technical effects.

I. Multiple angiography technologies applied. The Embodiment I of the gas-liquid preparation provided by the embodiment of the present disclosure was suitable for X-ray angiography and ultrasonic angiography which both could provide clear angiography images. The existing ultrasonic contrast agent was mainly a bubble with a thin and soft outer membrane, and the bubble was wrapped with high-density inert gas (insoluble in water or blood); and the diameter of the existing ultrasonic contrast agent was generally about 2-5 um, its stabilization time was long, and it had good vibration and echo characteristics, such as Option. Because the bubble-containing liquid had strong scattering characteristic on ultrasonic waves, the bubble-containing liquid serving as the ultrasonic contrast agent to be injected into the blood vessel of the human body would enhance the ultrasonic Doppler signal of the blood flow and improve the clarity and resolution of the ultrasonic image. Therefore, in case of CT, the image obtained through the liquid contrast agent such as ioversol was clearer than the image obtained through the gaseous contrast agent; and in case of B-ultrasonography, the image obtained through the gaseous contrast agent was clearer than the image obtained through the liquid contrast agent. In the Embodiment I of the present disclosure, the gaseous contrast agent and liquid contrast agent were combined, and the agents arranged in series at intervals were prepared by utilizing the property that the gaseous contrast agent and the liquid contrast agent were immiscible, thereby obtaining high-quality angiography images under CT or ultrasonic conditions.

II. The operation was simple and convenient. According to the embodiment of the present disclosure, the embolic agent, the liquid tracer agent and carbon dioxide were pre-filled into the conduit according to a set sequence, and the conduit was closed by the Luer taper. The contrast agent, the embolic agent and the perfusion agent could be simultaneously input through one-step operation (other drugs could also be input, and as the compatibility in the conduit and human tolerance were met, multiple drugs could be arranged at intervals in the conduit); the problems that the carbon dioxide could not be stored in clinic and a carbon dioxide manufacturing device was needed in the surgery were solved; a carbon dioxide manufacturing machine was not required in the surgery; the conduit filled with the tracer drug could be directly used; the multiple conduits could be interconnected through the Luer tapers; the drug dosage was not limited; and multiple types of drugs with large dosage could be input at a time.

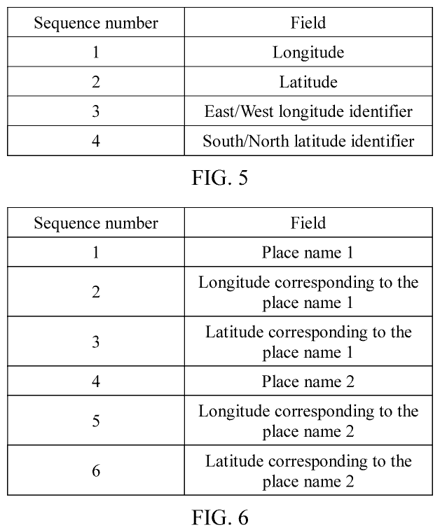

III. The drug dosage was saved. As shown in FIG. 5, after the first drug 11 at the head end entered the blood vessel, the second drug 12 that was oxygen immediately entered the blood vessel. As oxygen could be oxygenated with red blood cells, oxygen could be partially absorbed as long as the injection amount was not greater than 0.02 mL/kg (generally, air more than 0.02 mL/kg entering the blood vessel would make people uncomfortable, and air more than 2 mL/kg entering the blood vessel might cause sudden death). Taking coronary intervention treatment as an example, the injection amount of the contrast agent of a conventional dosage form was about 2 mL/kg, and the one-time injection amount was about 8 mL. If the gas-liquid preparation in the Embodiment I of the present disclosure was adopted, it was assumed that L1=L2, and only 4 mL of the first drug (contrast agent) was needed. This was because the target blood vessel could be fully filled with 8 ml of contrast agent in the prior art, and the conduit full of the gas-liquid preparation in the Embodiment I of the present disclosure would be used for delivering about 4 mL of contrast agent and 4 mL of oxygen to fully fill the target blood vessel. Therefore, the drug dosage could be saved by the present disclosure. It was noted that data in the present disclosure were only taken as examples to make understanding easy and did not cause any limitation on the present disclosure.

IV. The cell activity was increased, thereby improving the pharmaceutical effect. The oxygen could increase the activity of vascular endothelial cells and increase the absorption capacity of the endothelial cells to the embolic agents or other treatment agents in the gas-liquid preparation, and therefore, the spacing gas oxygen was injected at intervals while injecting the drugs so as to improve the pharmaceutical effect.

V. The anoxic environment of tumor cells was changed. In case of taking oxygen as the gas separant in the present disclosure, the anoxic state of tumors could be changed, and the tumor cells could be killed. Three scientists, the winners of 2019 Nobel Prize in Physiology or Medicine, found the mechanisms of Hypoxia-inducible factors (HIF) and biological oxygen perception pathways. Researches showed that the tumor cells induced hypoxia through various mechanisms to create a chronic anoxic environment, HIF signal pathways were activated, thus the growth of the tumors was accelerated, the invasiveness of the tumors was improved, and the tumors were promoted to be transferred. By utilizing the gas-liquid pharmaceutical preparation provided by the embodiment of the present disclosure, oxygen was delivered into tumor tissues, so the anoxic environment of the tumor cells was destroyed; and the pharmaceutical preparation could improve the effect of cancer treatment through the synergistic effect with radiotherapy and chemotherapy drugs.

VI. The concentration of the drugs was kept. After entering the blood vessel, the gaseous separant such as oxygen or carbon dioxide would extruded blood, so when entering the blood vessel, the drug behind the separant, namely the contrast agent (the contrast agent outside the contrast agent at the head end of the conduit), the embolic agent or the perfusion agent did not make contact with the blood, and was not diluted in the blood and did not cause a laminar flow phenomenon, and therefore the high concentration of the drugs could be kept (the concentration the same as that during injection). For example, the Embodiment II of the present disclosure was a circulation combination of liquid contrast agent-gaseous separant-embolic agent-gaseous separant, and the gaseous separant was carbon dioxide which had the separating effect and the tracing effect. In a vascular interventional embolization surgery, the tracing effect was effectively enhanced through the cooperation of the liquid contrast agent and carbon dioxide, the carbon dioxide separated the liquid tracer agent from the embolic agent, thereby preventing the concentration of the embolic agent from being affected by the blood while accurately and clearly tracing the embolic agent, and as a result, effective embolization was achieved.

VII. The oxygen was delivered during angiography or treatment to improve the comfort level of angiography or treatment. By adopting the contrast agent in the present disclosure, oxygen could be delivered to human tissues during angiography to improve the comfort level of the patient.

VIII. The flexibility of the drug compatibility was improved. As mentioned above, the drugs in the same conduit provided by the embodiment of the present disclosure could be separated through a proper separant, and thus the adjacent drugs on the two sides of the separant could be simultaneously injected into the human body (the compatibility requirement in the aspect of therapeutic effect was met). Therefore, the drugs which could not be combined together originally were relatively fixed between the separants due to the existence of the separants, and thus the drugs which could not be combined together could not be physically or chemically changed together. Therefore, on the premise of meeting the requirement in the aspect of therapeutic effect compatibility, the compatibility requirement of the agent in the aspect of physics or chemistry was reduced in the present disclosure.

IX. Accurate hemodynamic analysis would be conveniently performed. The hemodynamic analysis could be performed based on the image effect shown in FIG. 5. The gas-liquid preparation provided by the embodiment of the present disclosure contained oxygen, and oxygen bubbles could be used for measuring hemodynamic indexes; and the hemodynamic indexes could also be measured by monitoring the movement track of the contrast agent section by section. This was because the flow velocity, direction, quantity and the like of chain bubbles or liquid sections could be directly monitored. Moreover, in case of injecting through the injection pump 100, the injecting pressure could be detected. The injecting pressure was related to blood viscosity, vascular embolism state, concentration of the contrast agent/embolic agent and other factors, and more accurate vascular embolism data could be obtained through hemodynamic analysis and calculation in combination with the data of the angiography image, thus the therapeutic effect was improved, and the development requirements of the current medical big data technology were satisfied.

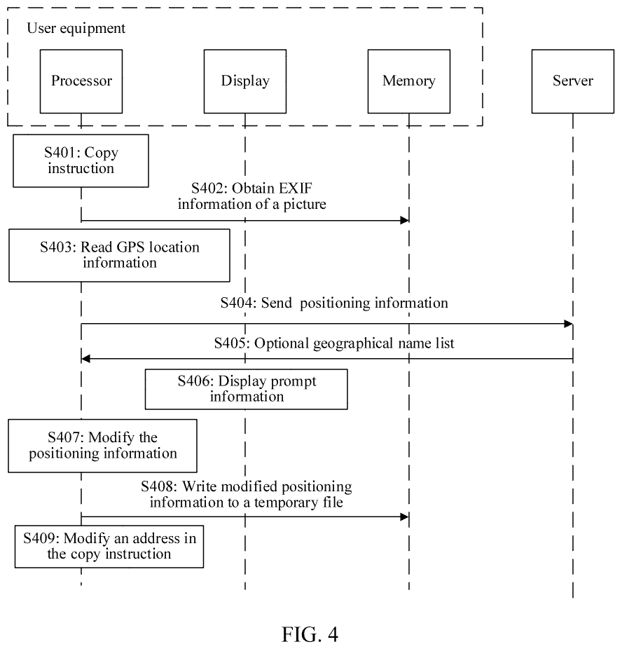

As shown in a schematic diagram in FIG. 4, by adopting the gas-liquid preparation provided by the embodiment of the present disclosure, the angiography image showed that blood vessels were chain-shaped, and the first drug 120 was the liquid contrast agent and was black; and the second drug 121 was oxygen and was light-colored bubbles, as shown in an experimental image in FIG. 5.

The gas-liquid preparation provided by the embodiment of the present disclosure could be clinically injected as the contrast agent by one step, then B-ultrasonic examination and CT examination were carried out in sequence, and it was not needed to inject the contrast agent respectively before the B-ultrasonic examination and the CT examination. In the present disclosure, the dosage of the contrast agent was reduced and the pain of the patient was relieved by being compared with the conventional contrast agent.

In order to verify the technical effects of the present disclosure, the inventor carried out the following experiments to observe the physical characteristics and the developing effect of the gas-liquid series embolic agent provided by the embodiment of the present disclosure.

I. Experimental Materials:

Carbon dioxide-alcohol gas-liquid series embolic agent: 10 polyethylene transparent conduits (polyethylene conduit, hereinafter referred to as PE conduit) pre-filled with the carbon dioxide-alcohol gas-liquid series embolic agent were provided, and each conduit was 100 cm in length and 2.0 mm in inner diameter; joints at two ends were standard male and female Luer tapers, which could be directly connected with the matched conduit joints; the pre-filled contents in the PE conduits were 75% alcohol solution and carbon dioxide; a computer was used for controlling a micro-flow pump valve to pre-fill at intervals; the unit length of each section of gas and liquid columns in the conduits could be controlled within 3-15 mm as required; the length of each section was controlled within 10 mm as much as possible in preparation of the 10 conduits in this batch; and the conduits were relatively uniform as much as possible. The 10 PE conduits were numbered according to the sequence of 10-1, 10-2 . . . 10-10.

II. Experimental Reagents and Devices:

(I) Reagents

1. Sodium chloride injection (250 ml: 2.25 g) produced by Guangdong Yixiang Pharmaceutical Co., Ltd.

2. An'erdian skin antiseptic III produced by Shanghai Likang Disinfection High-tech Co., Ltd.

(II) Experimental Devices:

1. X-ray photography system: German Siemens YISO DR digital X-ray photography system

2. Digital Subtraction Angiography System (DSA): German Siemens Axiom Artis dTA suspension digital flat-panel angiography system

3. Human body simulation model for X-ray photography

4. Self-made adjustable-length conduit X-ray photography frame.

At most 14 PE conduits could be fixed on the photography frame at the same time; 14 grooves were designed in a fixed plate at the upper end of the frame, the PE conduits were embedded into the grooves, the joints of the PE conduits were clamped at one of the ends of the grooves, and then the PE conduits were straightened; the other joints were clamped in grooves in another movable plate with the adjustable position, and 14 grooves were designed in the movable plate and corresponded to the grooves in the top end of the frame one to one; the position of the movable plate could be adjusted as a whole by using round holes in the two ends of the movable plate through threaded metal columns on the two sides of the photography frame; and after the photographed PE conduits were all straightened, two screws, at the upper and lower parts of the movable plate, on the metal columns were tightened, thereby ensuring that all the PE conduits were kept in the straightened state in the photographing process.

III. Experimental Method:

(I) Gross Observation of Gas-Liquid Series Embolic Agent

After the PE conduits were pre-filled, instant photos of samples were taken and observed; and after being subjected to commercial express delivery, the PE conduits were examined, observed and recorded.

(II) In-Vitro Digital X-Ray Photography of Gas-Liquid Series Embolic Agent

1. Method for fixing PE conduits

The head ends and the tail ends of 10 PE conduits containing carbon dioxide-alcohol were respectively fixed in the grooves in the upper and lower parts of the photography frame; the movable plate at the lower part was moved until the PE conduits reached the positions for realizing the maximum stretching state; the positions of two ends of the movable plate were fixed through the screws; the photography frame was erected in front of a detector; and it was prepared to carry out integral photographing on 10 embolic agent conduits.

2. Photographing method:

A digital image splicing method was adopted for photographing; on the basis of the projection distance of 180 cm, exposure was carried out in an upper section, a middle section and a lower section of an axis which was the middle point of each conduit; the photographing center line of the middle section was vertical to the middle point of the detector; the upper section and the lower section were respectively photographed by inclining the angle of a bulb tube; and three images were obtained after three times of exposure, and the images were transmitted to an image post-processing workstation for seamless splicing.

3. Photography parameters: three groups of different conduit voltages (kV) (49.90 kV; 80.90 kV; 89.80 kV) were adopted in photography, and the conduit current was independently adjusted through the system according to the conduit voltage (correspondingly, 772 mA; 916 mA; 909 mA).

(III) In-vitro DSA imaging of gas-liquid series embolic agent: perspective and subtraction photography acquisition

1. Preparation before DSA imaging: in order to simulate an angiography subtraction process during DSA subtraction photography acquisition, the 10-1 PE conduit was taken and straightened and then fixed on a rectangular wood board by medical adhesive tapes; and the 10-2 PE conduit was taken and straightened and then fixed on a rectangular hard paperboard, and then the conduit was fixed to a human abdomen simulation model in a long axis manner.

Preparation of an An'erdian-sodium chloride mixed solution: about 20 ml of sodium chloride injection was put into a flask, and a small amount of An'erdian skin antiseptic III was poured into the flask for staining, thus obtaining a yellowish-brown solution.

2. DSA imaging:

(1) The wood board on which the 10-1 PE conduit was fixed was put on a DSA examining table, and the developing conditions of gas and liquid in the 10-1 PE conduit were statically observed under perspective.

(2) The DSA examining table was fixed; the wood board on which the 10-1 PE conduit was fixed was pulled toward the head side at a constant speed by an assistant so as to move the PE conduit during perspective, thus the moving effect of the gas-liquid series embolic agent in the conduit could be simulated, and the developing effect of the embolic agent under perspective could be observed; and this process was repeated later, and DSA exposure photography was started for continuous subtraction acquisition.

(3) 5 ml of An'erdian-sodium chloride mixed solution was pumped by a 5 ml injector, the injector was connected with a 10-1 PE conduit joint, and the An'erdian-sodium chloride mixed solution was manually injected; carbon dioxide-alcohol in the PE conduit was pushed by using the mixed solution so as to be completely discharged from the conduits; therefore, the process of injecting the embolic agent from the interior of the PE conduit to the exterior of the conduit was simulated, the injection ended after observing that the PE conduit was full of the yellowish-brown An'erdian-sodium chloride mixed solution, and the scale of the injector at the moment was read, and it was at about 2 ml; and during injecting, the wood board on which the 10-1 PE conduit was fixed was kept static on the DSA table, the DSA table was fixed, exposure acquisition was performed for about 5 s to obtain a dynamic subtraction image, and the dynamic developing condition of the embolic agent conduit was observed.

(4) The above operation was repeated by the 10-2 PE conduit based on the human abdomen simulation model, and the DSA perspective and subtraction acquisition effects of the carbon dioxide-alcohol under the human body thickness condition were observed.

(IV) Observe the Change of Gas-Liquid Appearance in PE Conduits Pre-Filled with Carbon Dioxide-Alcohol

1. Storage condition and time: 10 PE conduits were packaged in a polypropylene (PP) material preservation box after arriving at a laboratory and were kept in a normal-temperature dry environment; two time points were randomly selected for observing the change of gas-liquid substances in the conduits at different time points in the environment: the time point of arriving at the laboratory for 1 week (recorded as time point 1), and the time point of arriving at the laboratory for 2 weeks (recorded as time point 2); and the overall change was indirectly reflected by observing and recording the length change of contents in the conduits at the two time points.

2. Gas-liquid unit length measurement: the length of contents in 9 PE conduits was measured (the 10-10 conduit was not counted due to the damage of joints in the experimental process). Each section of gas columns/liquid columns in the PE conduits was defined as a gas column/liquid column unit; digital X-ray photography was performed on the PE conduits when arriving at the laboratory for 1 week and 2 weeks respectively; the unit length of each gas column and liquid column formed by carbon dioxide and alcohol in the PE conduits was measured and recorded by using a linear measuring tool in a PACS, the unit was mm, and the numerical value was retained to the last two places of the decimal point; and the total length of the gas/liquid columns in the conduits was the sum of all the unit lengths of the gas/liquid columns in each conduit.

3. Statistical processing and data analysis:

Statistical analysis was performed by SPSS 16.0 statistical software, and all metering data were represented in an x±S form. Normality test was performed on the metering data by a Shapiro-Wilk method. The average numbers of two groups of independent samples were compared, and if the average numbers conformed to normal distribution, t test was performed; and if the average numbers did not conform to the normal distribution, rank sum test was performed; the average numbers of two groups of paired samples were compared, and if the average numbers conformed to the normal distribution, t test was performed; and if the average numbers did not conform to the normal distribution, symbol rank sum test was performed; multiple groups of quantitative data were compared, and if each group of data conformed to the normal distribution and had variance homogeneity, variance analysis processing was performed; and if each group of data did not conform to the normal distribution and did not have variance homogeneity, Welch's anova test was performed. There was statistical significance by taking P<0.05 as the difference.

IV. Experimental Result

(I) Gross Observation Result of Gas-Liquid Series Embolic Agent

10 conduits provided by the embodiment of the present disclosure were selected, the conduit bodies were transparent, gas columns and liquid columns which could be distinguished by naked eyes were filled at intervals in the conduits, and the unit length of the gas columns and the liquid columns was relatively uniform. FIG. 6A is the diagram showing matching of the gas-liquid preparation conduit head end joint and the gas-liquid preparation conduit tail end joint in the embodiment of the present disclosure. FIG. 6B was the state diagram of the conduit pre-filled with gas-liquid drugs in the embodiment of the present disclosure, and it could be seen that the unit length of the gas columns and liquid columns was relatively uniform.

(II) In-Vitro Digital X-Ray Photography Result of Gas-Liquid Series Embolic Agent

The gas-liquid series embolic agent was subjected to digital X-ray photography under three different conduit voltage conditions (49.90 kV; 80.90 kV; 89.80 kV), which could clearly display the arrangement of the gas columns and the liquid columns in the PE conduits; the window width and window position of the image could be properly adjusted to satisfy the requirements of experimental observation; and the picture showed that the components with high density in the conduits were liquid components (75% alcohol solution), and the components with low density in the conduits were gas components (carbon dioxide).

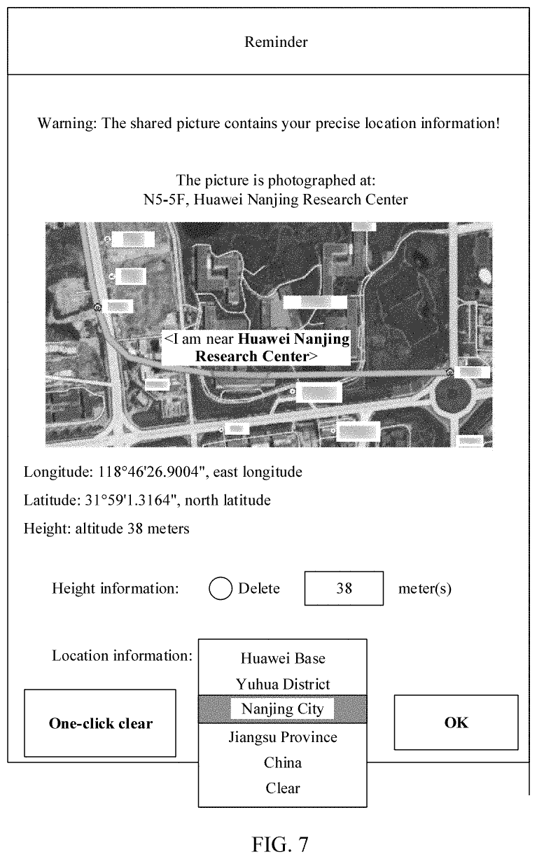

As shown in FIG. 7B, in case of using default conduit voltage of 80.90 kV, the image was grey, and the contrast display was still feasible and but was slightly worse than that in FIG. 7A; as shown in FIG. 7C, if the conduit voltage was increased to 89.80 kV, the grey degree of the image was further increased, and the contrast display condition was worse; and in case that the conduit voltage was properly reduced to 49.90 kV, the arrangement of the gas columns and the liquid columns in the PE conduit could be clearly displayed, and the image contrast was clear, as shown in FIG. 7A.

(III) In-Vitro DSA Imaging Result of Gas-Liquid Series Embolic Agent

1. The DSA examining table was fixed; the wood board on which the 10-1 PE conduit was fixed was pulled toward the head site at a constant through the assistant, only the appearance of the PE conduit could be observed under perspective, the motion condition of the PE conduit could not be clearly observed, and gas-liquid components in the conduit could not be distinguished; and the subtraction image acquired by continuous exposure through the DSA could clearly show the PE conduit and the interval characteristics of gas-liquid columns inside, it was light and shade alternation, the bright (white) was 75% alcoholic solution, the dark (grey black) was carbon dioxide, and moreover, the motion condition of the PE conduit could be observed. The gas-liquid column length intervals were unevenly arranged through observation by naked eyes.

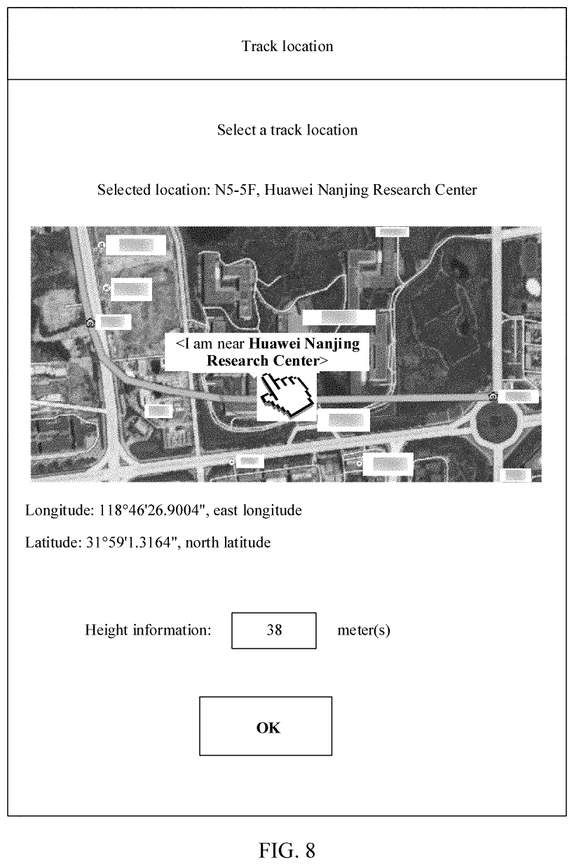

2. When injecting the An'erdian-sodium chloride mixed solution into the PE conduit, only the appearance of the PE conduit could be observed under the perspective condition, and the motion condition of the content and the gas-liquid components in the PE conduit could not be clearly observed; and as shown in FIG. 8A, continuous exposure acquisition was performed in the DSA subtraction state, thus the appearance of the PE conduit and the interval characteristics of the gas-liquid columns inside could be clearly displayed, and the forward injecting image of the carbon dioxide and the alcoholic solution in the PE conduit could also be clearly displayed.

3. Under the condition of adding the abdominal model, the 10-2 conduit was manually pushed to simulate advancing, and continuous exposure acquisition was performed in the DSA subtraction state so as to obtain the image; the image of the overall motion of the PE conduit could be observed, and the image composed of the carbon dioxide and the alcoholic solution at intervals in the PE conduit could be basically distinguished, as shown in FIG. 8B; and through the image obtained through continuous exposure acquisition in the DSA subtraction state when injecting the An'erdian-sodium chloride mixed solution into the PE conduits, the process that the contents in the PE conduit were pushed to move forwards for a short time could be observed, but the gas column and liquid column intervals were not clearly distinguished.

IV. Observation Result of Gas-Liquid State Change in PE Conduits Pre-Filled with Carbon Dioxide-Alcohol

(I) Compare the Unit Length of the Gas Columns and the Liquid Columns in 9 PE Conduits Arrived at the Laboratory for 1 Week and 2 Weeks Respectively

Result: 1. The unit length of the liquid columns in the 9 PE conduits was compared when arriving at the laboratory for 1 week, and the difference had statistical significance (P=0.00). 2. The unit length of the gas columns in the 9 PE conduits arrived at the laboratory for 1 week was compared, and the difference had statistical significance (P=0.00). 3. The unit length of the liquid columns in the 9 PE conduits arrived at the laboratory for 2 weeks was compared, and the difference had statistical significance (P=0.00). 4. The unit length of the gas columns in the 9 PE conduits arrived at the laboratory for 2 weeks was compared, and the difference had statistical significance (P=0.00).

(II) Compare the Unit Length of the Gas Columns and the Liquid Columns in Each of the 9 PE Conduits Arrived at the Laboratory for 1 Week and 2 Weeks Respectively

Result: 1. The difference obtained by comparing the unit length of the gas columns and the liquid columns in the conduits had statistical significance (P<0.05) in case that the conduits 10-1, 10-2, 10-7 and 10-8 were at the two points and the conduit 10-5 arrived at the laboratory for 2 weeks, and the unit length of the liquid columns was greater than the unit length of the gas columns; and 2. The difference obtained by comparing the unit length of the gas columns and the liquid columns in the conduits did not have statistical significance (P>0.05) in case that the conduits 10-3, 10-4, 10-6 and 10-10 were at the two points and the conduit 10-5 arrived at the laboratory for 1 week.

(III) Compare the Unit Length of the Gas Columns/Liquid Columns in the 9 PE Conduits Arrived at the Laboratory for 1 Week and 2 Weeks

Result: 1. The unit length of the liquid columns in the 9 PE conduits arrived at the laboratory for 1 week and 2 weeks was compared, and the difference did not have statistical significance (P=0.338); and 2. The unit length of the gas columns in the 9 PE conduits arrived at the laboratory for 1 week and 2 weeks was compared, and the difference did not have statistical significance (P=0.055).

(IV) Compare the Total Length of the Gas Columns and the Liquid Columns in the 9 PE Conduits Arrived at the Laboratory for 1 Week and 2 Weeks Respectively

Result: 1. The difference obtained by comparing the total length of the gas columns and the liquid columns in the 9 PE conduits arrived at the laboratory for 1 week had statistical significance (P=0.041), and the total length of the liquid columns in the conduits was greater than that of the gas columns in the conduits; and 2. The difference obtained by comparing the total length of the gas columns and the liquid columns in the 9 PE conduits arrived at the laboratory for 2 weeks had statistical significance (P=0.039), and the total length of the liquid columns in the conduits was greater than that of the gas columns in the conduits.

(V) Compare the Total Length of the Gas Columns and the Liquid Columns in the 9 PE Conduits Arrived at the Laboratory for 1 Week and 2 Weeks

Result: 1. The total length of the gas columns in the 9 PE conduits arrived at the laboratory for 1 week and 2 weeks was compared, and the difference did not have statistical significance (P=0.632); and 2. The total length of the liquid columns in the 9 PE conduits arrived at the laboratory for 1 week and 2 weeks was compared, and the difference did not have statistical significance (P=0.072).

The comparison of the unit length of the gas columns and the liquid columns in the conduits was shown in Table 1, and the comparison of the total length of the gas columns and the liquid columns in the conduits was shown in Table 1.

TABLE 1
Comparison of unit length of gas columns
and liquid columns in conduits (unit: mm)

| | Unit length of | | Unit length of | |
|---|---|---|---|---|
| Conduit | liquid column (mm) | | gas column (mm) | |
| No. | Time point 1 | Time point 2 | Time point 1 | Time point 2 |
| 10-1 | 8.80 ± 7.67 | 10.21 ± 9.00 | 4.99 ± 3.36 | 6.25 ± 5.74 |
| 10-2 | 8.13 ± 6.37 | 10.62 ± 8.78 | 3.40 ± 2.15 | 4.07 ± 3.91 |
| 10-3 | 5.21 ± 4.07 | 6.67 ± 4.01 | 6.06 ± 5.14 | 7.92 ± 5.69 |
| 10-4 | 6.09 ± 4.76 | 5.84 ± 6.39 | 5.06 ± 2.98 | 4.77 ± 3.07 |
| 10-5 | 6.94 ± 4.32 | 6.56 ± 3.54 | 5.95 ± 4.94 | 5.68 ± 5.29 |
| 10-6 | 5.97 ± 4.58 | 6.50 ± 5.01 | 5.90 ± 5.04 | 6.17 ± 3.83 |
| 10-7 | 9.10 ± 5.20 | 8.98 ± 5.78 | 6.28 ± 4.93 | 6.34 ± 4.17 |
| 10-8 | 7.86 ± 6.44 | 7.48 ± 6.90 | 4.85 ± 3.20 | 4.59 ± 2.88 |
| 10-9 | 7.31 ± 5.21 | 7.60 ± 5.35 | 7.58 ± 5.70 | 7.58 ± 4.39 |

TABLE 2
Comparison of total length of gas columns
and liquid columns in conduits (unit: mm)

| | Total length of gas columns | Total length of liquid columns |
|---|---|---|
| | in conduits (n = 9) | in conduits (n = 9) |
| Time point 1 | 416.47 ± 71.77 | 539.39 ± 81.36 |
| Time point 2 | 420.07 ± 80.15 | 548.46 ± 80.17 |

V. Result Analysis

(I) In-Vitro Digital X-Ray Photography of PE Conduits Pre-Filled with Gas-Liquid Series Gas-Liquid

X-ray photography was performed on 10 conduits pre-filled with the gas-liquid series embolic agent; and it could be clear to distinguish different gas and liquid components in the conduits, the carbon dioxide gas component showed low density, the absolute alcohol solution showed relatively slightly high density, the gas-liquid intervals of the reagents in the conduits could be clearly displayed according to the density difference, and thus a satisfactory X-ray shot picture could be obtained. In this experiment, the PE conduits were long, so it was needed to utilize the digital X-ray photography splicing technology to obtain a complete shot picture covering the whole conduits. The digital X-ray splicing photography was mainly used for photographing spinal columns and bone joints in clinic, aiming to obtain a complete anatomical structure image. The X-ray photography in this experiment adopted a mode of inclining the angle of the bulb tube, thus obtaining an ideal spliced image.

(II) In-Vitro Digital Perspective and Subtraction Acquisition Observation on Gas-Liquid Series Embolic Agent

In this experiment, the PE conduits pre-filled with gas-liquid were observed by the DSA device utilizing a perspective mode and a subtraction exposure acquisition mode. The image obtained by subtraction acquisition after fixing the PE conduits on the wood board and integrally moving the wood board to simulate the movement of the embolic agent had the distinguishing effect obviously better than that obtained by perspective; and in fact, the pre-filled carbon dioxide, alcohol and the like in the PE conduits were not changed, so the image acquired by simulating the DSA could be used for clearly distinguishing different components of gas and liquid in the PE conduits, and the simulated dynamic subtraction image could also clearly display the gas and liquid.

(III) Change of Gas and Liquid Components in Gas-Liquid Series Embolic Agent after Transporting and Storing

9 PE conduits pre-filled with gas-liquid were observed at different storage time points in short time after being transported, the unit length of gas and liquid in each conduit stored for 1 week and 2 weeks after transportation was respectively compared, and the results both showed statistical differences, so it could be considered as that the unit length of gas and liquid of the contents in the 9 PE conduits pre-filled with gas-liquid had difference at the two time points. It was prompted that the length of gas and liquid columns in the PE conduits changed, namely, the gas and liquid were mixed.

The change of the unit length of the liquid columns in the 9 PE conduits pre-filled with gas-liquid and stored for 1 week after transportation was independently compared, and the result showed that the difference did not have statistical significance; the unit length of gas in the PE conduits was independently compared, the result also showed that the difference did not have statistical significance, and it indicated that the volume of liquid and gas pre-filled in the PE conduits stored for one week after transportation did not decrease; and the alcohol volatilization amount could be ignored. The unit length of the gas and liquid in each PE conduit pre-filled with gas-liquid and stored for 1 week and 2 weeks after transportation was compared, and the result showed that 1 PE conduit stored for 2 weeks had the difference with statistical significance; 4 PE conduits did not have the difference with statistical significance, and another 4 PE conduits had the difference with statistical significance; and the latter showed that the total length of the liquid columns was larger than that of the gas columns, and this was because the gas component in part of the conduits was reduced.

After being delivered by express, the 9 PE conduits pre-filled with gas-liquid in original packaging bags were stored in the PP material preservation box and placed in a 20° C. dry environment. The change of gas and liquid components in the PE conduits pre-filled with gas-liquid might be caused by the following factors: 1. Part of carbon dioxide was dissolved in the alcoholic solution at normal temperature, the carbon dioxide could react with water to generate carbonic acid, the generated carbonic acid was unstable and could be decomposed into water and carbon dioxide again, and the chemical reaction between the carbon dioxide and the water was reversible, so the carbon dioxide gas in the conduits could not be kept in an absolutely stable state; 2. The sealing property of sealing nuts of the joints at two ends of the PE conduits pre-filled with the gas-liquid series embolic agent was designed for common liquid, and the gas and volatile liquid could not be completely sealed; and 3. The position change of gas and liquid units could be caused by stretching and curling of reagent tubes in the experimental process. However, the change did not influence the tracing and embolization effects.

The following described the animal experiments of the pharmaceutical preparation of the present disclosure.

Purposes:

The experiment was to perform renal artery embolization experiment on experimental rabbits with the gas-liquid series embolic agents with different formulas, observe the operability of the pharmaceutical preparation provided by the embodiment of the present disclosure in the process of living body interventional surgery, and know the embolization effect of each gas-liquid series embolic agent through pathological observation.

I. Experimental Animals:

2 male healthy ordinary New Zealand rabbits were selected from Huadong Xinhua Experimental Animal Farm, Huadu District, Guangzhou City (license number: SOCK (YUE) 2019-0023), named as an experimental rabbit 1 and an experimental rabbit 2 in the order of experiments and weighed as 2.0 kg (experimental rabbit 1) and 2.1 kg (experimental rabbit 2) respectively. The rabbits were raised in one cage for 2-3 d to adapt to the environment.

II. Main Reagents, Apparatuses and Devices

(I) Reagents:

1. There were 5 kinds of gas-liquid series embolic agents, and the pre-filled components and numbers were shown in Table 3 below:

TABLE 3
Gas-liquid series embolic agent PE conduit and pre-filled content

| | | Conduit |
|---|---|---|
| Number | Pre-filled content of PE conduits | length |
| 4-1 | CO₂ + 75% C₂H₆O (carbon dioxide + 75% alcohol) | 100 cm |
| 4-2 | CO₂ + 75% C₂H₆O (carbon dioxide + 75% alcohol) | 100 cm |
| 4-3 | CO₂ + 75% C₂H₆O + Na₂CO₃ (carbon dioxide + | 100 cm |
| | 75% alcohol + sodium carbonate) | |
| 4-4 | CO₂ + 75% C₂H₆O + Na₂CO₃ (carbon dioxide + | 100 cm |
| | 75% alcohol + sodium carbonate) | |
| 4-5 | CO₂ + C₂H₆O (carbon dioxide + pure absolute | 100 cm |
| | alcohol) | |

2. Su-Mian-Xin injection II (2 ml: 0.2 g): Dunhua Shengda Animal Medicine Co., Ltd.

3. 1% pentobarbital sodium injection: 1 g of pentobarbital sodium powder was dissolved in 100 ml of normal saline to prepare a 1% pentobarbital sodium solution

4. Iodixanol (320 mgI/ml): GE Corporation, USA, trade name: Visipaque

5. Heparin sodium injection (2 ml: 12,500 U): Chengdu HEPATUNN Pharmaceutical Co., Ltd.

6. Lidocaine (5 ml: 0.1 g): Shanghai Zhaohui Pharmaceutical Co., Ltd.

(II) Apparatuses:

6F radial artery puncture kit (AVANTI): 504-616Z, Johnson & Johnson, USA

5F KMP conduit: HNB5.0-38-40-P-NS-KMP, COOK MEDICAL LLC, USA

2.7F micro-conduit (Progreat): MC-PE27131, Terumo Corporation, Japan

(III) Devices:

1. Digital subtraction angiography system (DSA): Germany Siemens Axiom Artis dTA suspension digital flat-panel angiography system

2. Self-made adjustable-length conduit X-ray photography frame

III. Experimental Method

(I) Digital X-Ray Photography on Gas-Liquid Series Embolic Agent and Estimation of Volume of Liquid in PE Conduits

Before the animal embolization experiment, all reagent tubes to be used in this experiment were subjected to digital X-ray photography. The embolic agent conduits were fixed on the self-made conduit X-ray photography frame, and the photography was still performed by the digital image splicing method (see the experiment above).

The length of the liquid columns in the conduits in the obtained X-ray shot picture was measured by the same method, and a linear measuring tool arranged in the PACS was also utilized; the accumulated length of the liquid columns in each conduit was the total length of the liquid columns in each conduit, and the inner diameter of each PE conduit was 2.0 mm; and the volume of the liquid in each PE conduit was estimated by a cylindrical volume formula.

V=πr²h

(II) Embolization of Renal Artery Through Right Carotid Artery Access

1. Experimental Embolization with CO₂+C₂H₆O

The experimental rabbit 1 was taken and was intramuscularly injected with 0.2 ml of Su-Mian-Xin II through the left hindquarter muscle and intravenously injected with 2.5 ml of 1% pentobarbital sodium through the left side ear margin for composite anesthesia. After the anesthesia was successful, the rabbit lay on the DSA examining table in a foot-head position, the limbs were fixed on a self-made experimental board, and the right neck was subjected to skin preparation, disinfection and towel laying. The right carotid artery pulsation part was found along the right edge of the trachea, the skin was incised, and the subcutaneous tissue was separated layer by layer to expose the right common carotid artery. 2 silk threads were introduced front the lower part of the right common carotid artery and were respectively fixed at the upper and lower parts of an artery puncture point; a small amount of 1% lidocaine was locally sprayed for infiltration; the assistant gently lifted the silk threads on two sides to fix the right common carotid artery, a 21 G radial artery puncture trocar was used for puncturing the front wall of the artery, a needle core was withdrawn after the puncture was successful, and a guide wire was introduced; a puncture needle sheath was withdrawn after the guide wire was confirmed to be within the artery stroke through perspective; a 6F conduit sheath was introduced along the guide wire; and the conduit sheath was ligatured and fixed by the silk thread at the lower part of the puncture point, and the silk thread at the upper part of the puncture point was used for ligaturing the carotid artery to prevent bleeding. 5 ml of 0.1% heparin saline was injected through a bypass of the conduit sheath to prevent the conduit sheath from thrombus. A KMP conduit was introduced to the abdominal aorta through the guide wire, and the contrast agent that was iodixanol was manually injected for angiography.

The middle lower pole branch in the left renal artery was selected as a target branch for embolization; a micro-guide wire was used for guiding the micro-conduit to enter the target branch; and 1 ml of iodixanol was slowly injected under perspective monitoring to confirm that the micro-conduit was in the target branch, the 4-5 PE conduit (CO₂+C₂H₆O) was connected to the tail end of the micro-conduit, and the rear end of the PE conduit was connected with the injector to manually inject 2.5 ml of iodixanol. Carbon dioxide+absolute alcohol in the PE conduit were slowly and completely pushed out; and after 5 min, the iodixanol was used for reexamining target branch angiography, showing that the end branch of the target branch was reduced. In the experiment process, 2 ml of 1% pentobarbital sodium was added to keep the experimental rabbit sedative. The micro-conduit was introduced into the right renal artery trunk for angiography, showing that the diameter of the right renal artery trunk was spasmodically thinned, so the embolization test was not performed on the right renal artery.

2. Experimental Embolization with CO₂+75% C₂H₆O+Na₂CO₃

The experimental rabbit 2 was taken; and the left renal artery was treated as the target artery. The experimental steps were the same as above: the experimental rabbit was anesthetized and disinfected, the right carotid artery was exposed and punctured, and the KMP conduit was introduced to the abdominal aorta for angiography. The micro-conduit entered the left renal artery trunk, 1 ml of iodixanol was injected under perspective monitoring, the gas-liquid series embolic agent 4-3 conduit (CO₂+75% C₂H₆O+Na₂CO₃) was connected to the tail end of the micro-conduit, and the rear end of the embolic agent conduit was connected with the injector to manually inject 4 ml of iodixanol. Then, the gas-liquid series embolic agent 4-4 conduit (CO₂+75% C₂H₆O+Na₂CO₃) with the same components was injected by the same way; the left renal artery was also treated as the target artery; and the rear end of the embolic agent conduit was connected with the injector to manually inject 3 ml of contrast agent.

3. Experimental Embolization with CO₂+75% C₂H₆O

The experimental rabbit 2 was continuously used; and the right renal artery was treated as the target artery. The micro-conduit entered the right renal artery trunk, 1 ml of iodixanol was firstly injected under perspective monitoring; the rear end of the micro-conduit was connected with a gas-liquid series embolic agent 4-1 PE conduit (CO₂+75% C₂H₆O), and the rear end of the PE conduit was connected with the injector to inject 3 ml of iodixanol; and the developing condition of the gas-liquid series embolic agent under the condition of taking iodixanol as a reference was observed during injection. Then the gas-liquid series embolic agent 4-2 conduit (CO₂+75% C₂H₆O) with the same components was directly injected by using 3 ml of iodixanol; and the right renal artery was also treated as the target artery. DSA was immediately reexamined after the right renal artery was embolized, showing that the development of the distal branch of the right renal artery disappeared.

After the left and right renal artery embolization of the experimental rabbit 2 was completed, the micro-conduit was guided to enter the left renal artery trunk to be subjected to reexamining angiography through iodixanol, and there was no development of the distal branch of the left renal artery. The micro-conduit was placed in the abdominal aorta for angiography reexamining after 5 min, showing that there was still development of the right renal artery and most branches.

(IV) Pathological Examination:

The experimental rabbit 1 and the experimental rabbit 2 were immediately killed after the embolization was completed; the experimental rabbits were dissected, and both kidneys were taken out for appearance change observation; then kidney specimen on the embolization side was put into a 10% formalin solution and fixed for 12 h; both kidney specimens were taken out on the next day and cut from the renal portal along the coronal plane and the cross section respectively, and four parts were obtained; the kidney specimen on each side was divided into an upper abdominal side, an upper dorsal side, a lower abdominal side and a lower dorsal side; then the specimens were wrapped with paraffin; conventional HE staining and elastic fiber staining were performed on the specimens; and the specimens were observed under an optical microscope, and pathological changes were recorded.

Experimental Results

I. Estimation Result of Volume of Liquid in PE Conduits

The estimated results of the volume of liquid components in each PE conduit were shown in Table 4.

TABLE 4
Estimation of volume of liquid in PE conduits

| Factory | Total length of liquid | Estimated value of volume of |
|---|---|---|
| number | columns in conduits (cm) | liquid in conduits (ml) |
| 4-1 | 59.13 | 1.86 |
| 4-2 | 78.76 | 2.47 |
| 4-3 | 75.37 | 2.37 |
| 4-4 | 77.32 | 2.43 |
| 4-5 | 77.08 | 2.43 |

II. Pathological Observation Results

(I) External Observation Performance:

1. Experimental Rabbit 1:

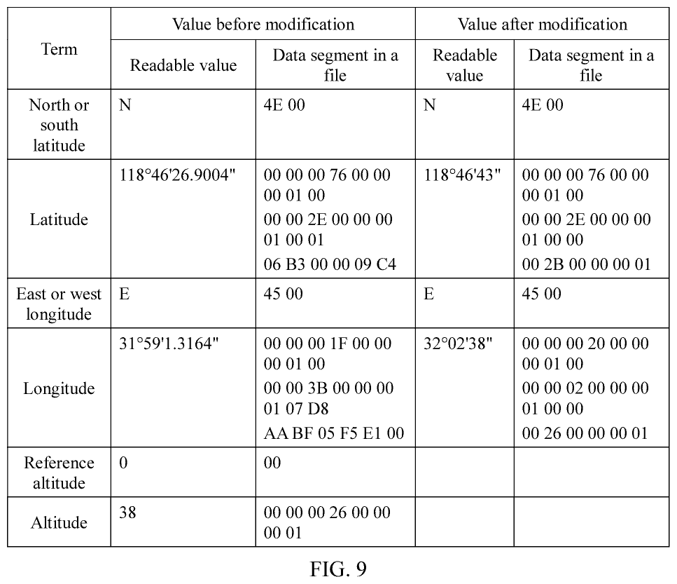

After embolization, immediate observation was performed and showed that the kidney (left kidney) on the embolization side swelled, its long diameter was about 3.4 cm, and its transverse diameter was about 2.2 cm; the long diameter of the kidney (right kidney) on the non-embolization side was about 3.1 cm, and the transverse diameter was about 2.1 cm; and the surfaces of the kidneys were still smooth, and an ischemic region, namely the middle lower part of the left kidney, changed in a large-flake dark yellow mode (FIG. 9A).

After being soaked in formalin for 12 h, the specimens were taken out for observing, the volume of the kidney (left kidney) on the embolization side was basically the same as above, its long diameter was about 3.4 cm, and its transverse diameter was about 2.1 cm; and the corticomedullary differentiation of the kidney (left kidney) on the embolization side was clear, and the ischemic region at the middle lower part changed in a grayish yellow mode (FIGS. 9B and 9C).

2. Experimental Rabbit 2:

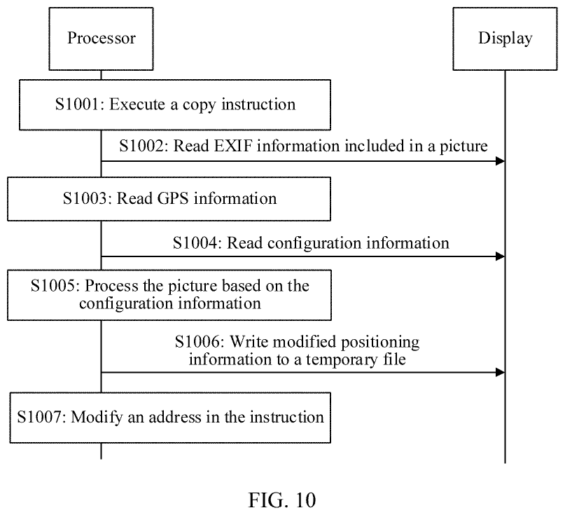

After embolization, immediate observation was performed and showed that both kidneys swelled, the long diameter of the left kidney was about 2.8 cm, and the transverse diameter was about 1.8 cm; the long diameter of the right kidney was about 2.9 cm, and the transverse diameter was about 1.9 cm; and the surfaces of both kidneys were still smooth, and both kidneys had scattered patch or spot-shaped dark yellow ischemic regions (FIG. 10A).

After being soaked in formalin for 12 h, the specimens were taken out for observing: the volume of both kidneys was larger than the previous volume, the long diameter of the left kidney was about 3.1 cm, and the transverse diameter was about 2.0 cm; the long diameter of the right kidney was about 3.3 cm, and the transverse diameter was about 2.2 cm; and the corticomedullary differentiation of both kidneys were still clear, and both kidneys were in uneven grayish yellow ischemic change (FIGS. 10B, 10C and 10D).

(II) Performance Under Microscope:

1. Experimental Rabbit 1:

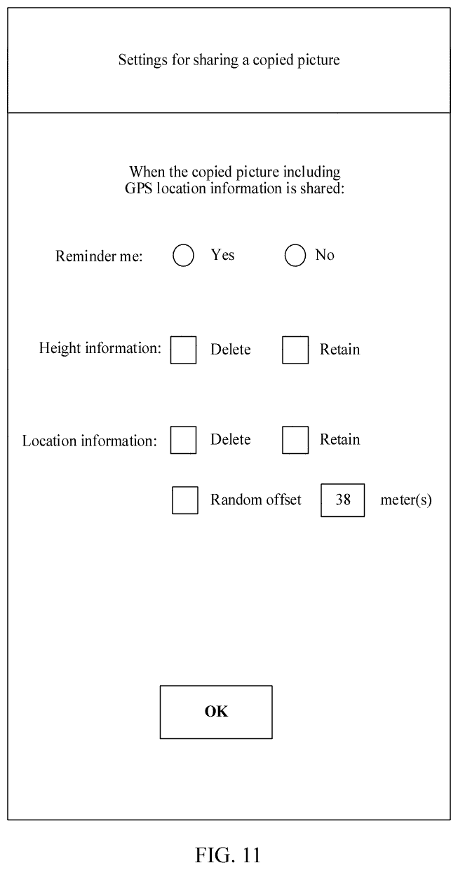

HE staining: it could be observed from the upper dorsal side of the left kidney that the tubular epithelial cells were subjected to edema degeneration, and the arteriolar wall was subjected to edema; and glomerulus and renal interstitium did not show clear pathological changes (FIG. 11A).

Elastic fiber staining: it could be observed from the upper dorsal side of the left kidney that extremely individual arterial wall was subjected to elastic fiber breakage (FIG. 11B).

2. Experimental Rabbit 2:

HE staining: (1) There were several small focal renal cortex infarction regions on the upper dorsal side of the right kidney, the renal tubular cells were subjected to edema degeneration, brush-like edges of proximal convoluted tubular epithelial cells disappeared, lightly-stained particles appeared in cell cytoplasm, and the cells were subjected to particle degeneration change; glomerular cell nucleuses in the infarction regions were fragmented and dissolved, and there was a change after cell necrosis; and there was no clear pathological change in renal interstitium (FIG. 12A). (2) Some tubular epithelial cells of the left kidney were subjected to edema degeneration; and there was no clear pathological change in glomerulus and renal interstitium (FIG. 12B).

III. Discussion on Pathological Effects of Three Different Tracing Pharmaceutical Preparations on Kidneys

In this experiment, it was the first time to use carbon dioxide as the contrast agent, three alcohol-based chemical embolic agents were respectively carried to enter renal arteries, and pathological changes of absolute alcohol, 75% alcohol+sodium carbonate and 75% alcohol on the renal arteries and renal tissues were preliminarily observed.

(I) Absolute Alcohol

In this experiment, the left kidney of the rabbit 1 was injected with carbon dioxide+absolute alcohol through renal artery, large-area ischemic change could be observed immediately by naked eyes, but corresponding renal cortex cell necrosis was not found under the microscope; after discussion with doctors of the pathology department, it was speculated that the time from absolute alcohol injection to specimen acquisition and fixation was short, and the cells in the renal ischemic region were not subjected to necrosis change; the renal tubule was more sensitive to ischemia, so the pathological change under the microscope was mainly concentrated on the renal tubule, showing degenerative edema on the tubular epithelial cells; and the edema of arteriolar wall might be related to the damage caused by absolute alcohol, the change of elastic fiber breakage of part of the arterial wall was not a common phenomenon under the microscope, and it could not be determined whether it was the consequence of the absolute alcohol or the degeneration of the rabbit vascular wall at present.

(II) 75% Alcohol

In the experiment, it was tried to inject 75% alcohol through renal artery, and the specimen was taken immediately after embolization for pathological observation. According to the observation result, renal tubular epithelium denaturation and arteriolar wall edema occurred after injecting 75% alcohol through renal artery, even focal infarction change occurred in one kidney, and its pathological change was similar to that caused by absolute alcohol. 75% alcohol was used for disinfection, and its mechanism included: (1) hypertonic dehydration of bacterial cells was caused, alcohol molecules could act on the peptide chain link of protein molecules to cause protein denaturation and precipitation, and this effect was more obvious when the content was 70%; and (2) 60-85% alcohol could easily permeate into the bacteria to destroy and dissolve the bacterial cells; and

(3) the alcohol had a destructive effect on a microbial enzyme system: the alcohol inhibited normal metabolism by inhibiting a bacterial enzyme system, especially oxidase, dehydrogenase and the like, so as to inhibit bacterial growth.

(III) 75% Alcohol Mixed with Sodium Carbonate

In this experiment, it was innovative to add sodium carbonate into 75% alcohol solution, aiming to enable sodium carbonate to react with water in the solution to generate carbonate, and thus the embolic agent contained alkaline components to realize embolization with acid-base balance. However, the healthy rabbits were used as the test objects in the experiment, and the preliminarily obtained result was similar to that of only using pure 75% alcohol and absolute alcohol.

In conclusion, three vascular intervention tracing pharmaceutical preparations in the experimental formula could cause degeneration and necrosis of renal tubular cells and cause edema of the arteriolar wall.

### Experiment II

Purposes:

2.1 The purpose was to observe the visibility of the gas-liquid embolic agent with the ratio under perspective, and determine whether the flowing direction, stagnation or backflow of gas-liquid flow could be clearly observed.

2.2 The purpose was to observe the influence of the gas-liquid embolic agent with the ratio on local blood flow after injecting into blood vessels, such as slow blood flowing, blood flow stagnation or interruption, and

DSA and ultrasonic image data of local blood vessels after embolization.

2.3 The purpose was to find out the optimal gas-liquid embolic agent ratio and embolic agent dosage according to the experimental result.

III. Observation of Experimental Process

3.1 The left renal artery of the experimental rabbit was selected, the carbon dioxide contrast agent was injected by using normal saline, the running condition of the gas columns passing through the artery-parenchyma was dynamically monitored by using B-ultrasound, and the following information was observed and recorded:

(1) Blood flow velocity change.

(2) Carbon dioxide absorption condition.

(3) Basic recovery time of blood flow.

3.2 The left and right kidneys were alternatively subjected to injection according to the situation.

3.3 After carbon dioxide was completely absorbed, pre-embolization angiography was performed on the super-selected left renal artery, and the alcohol embolic agent was injected and was manually slowly injected for DSA observation; and after entering the target blood vessel, the gas-liquid embolic agent gradually and slowly flowed until stopping or being subjected to a small amount of backflow, the clarity of the process under perspective was observed, and the dosage of the embolic agent injected into the target blood vessel was recorded.

3.4 Angiography observation was performed immediately after the embolic agent injection was completed, and angiography observation was respectively performed on the target blood vessel and the branch embolic result thereof after 5 min, 15 min and 30 min.

3.5 Alcohol embolic agent observation was sequentially performed on the right kidney and liver, and data was further collected.

3.6 Alcohol embolic observation was sequentially performed on the common carotid artery and the external carotid artery.

3.7 The specimens of the kidney and liver of the experimental rabbit were prepared for pathological analysis after the embolization experiment was completed.

V: Experimental Process

5.1 On Aug. 4, 2020, the first novel embolic agent animal experiment was conducted.

5.1.1 Sample details of gas-liquid embolic agent:

Carbon dioxide contrast agent: 2 pcs;

the conduit was made of Teflon, with an inner diameter of 2 mm, an outer diameter of 2.4 mm, and a length of 1,000 mm; and

the filler was shown in the table below.

| Sample | | Volume | Dosage for | Volume of |
|---|---|---|---|---|
| details | Quantity | of CO₂ | contrast agent | 75% alcohol |
| 75% alcohol- | 6 pcs | 20-50 μl for | 15 μl for | 40 μl for |
| CO₂-80% | | single | single | single |
| ioversol | | section | section | section |

5.2.2 The gas-liquid preparation used in this experiment was the contrast agent diluted to 75%, and it could be clearly displayed under perspective.

5.2.3 Carbon dioxide-saline-contrast agent was injected through the left renal artery, 30 μl of carbon dioxide entered the blood vessel, and the gas was basically absorbed and the blood flowed normally by ultrasonic observing after 3 min.

5.2.4 Pre-embolization angiography was performed on the left renal artery; a novel embolic agent was injected; the injector scale was at 1.5 ml when the embolic agent started to enter the blood vessel, and the injector scale was at 0.8 ml when blood backflow occurred, so it was calculated that 0.7 ml of embolic agent entered the target blood vessel in total, including about 350 μl of 75% alcohol and about 240 μl of carbon dioxide; the observation image was clear under DSA; and the contrast agent was introduced 5 min after injection, showing that the whole kidney was basically embolized.

5.2.5 Pre-embolization angiography was performed on the right renal artery; the novel embolic agent was injected, the injector scale was at 2.0 ml when the embolic agent started to enter the blood vessel, the injector scale was at 1.2 ml when blood backflow occurred, so it was calculated that 0.8 ml of the embolic agent entered the target blood vessel in total, including about 300 μl of 75% alcohol and about 300 μl of carbon dioxide; the observation image was clear under DSA; the contrast agent was introduced 5 min after injection; therefore, kidney blood vessel end embolization was performed more thoroughly; and a small amount of alcohol entered distal vein.

5.2.6 Carbon dioxide-saline-contrast agent was injected through hepatic artery, and continuous bubbles appeared in portal vein after 2.5 min.

5.2.7 The embolic agent was injected through hepatic artery, and 1.2 ml of embolic agent was injected after blood flowed back, including about 500 μl of 75% alcohol and about 600 μl of carbon dioxide; and the embolization condition was observed by injecting the contrast agent after 5 min, showing that the hepatic artery was completely embolized.

5.2.8 The embolic agent was injected through carotid artery, and 1.2 ml of embolic agent was injected after blood flowed back, including about 500 μl of 75% alcohol and about 600 μl of carbon dioxide. The embolization condition was observed by injecting the contrast agent after 5 min, showing that the carotid artery was completely embolized.

5.2.9 Conclusion:

5.2.9.1 The gas-liquid embolic agent was injected through the hepatic artery, continuous bubbles appeared in portal vein, and further experiment was needed to find out the reason of this phenomenon.

5.2.9.2 Angiography observation was not performed immediately, 5 min and 10 min after the liver, kidney and external carotid artery were embolized, and the specific reason of embolization was not eliminated.

5.3 On Aug. 20, 2020, the third novel embolic agent animal experiment was conducted.

5.3.1 Sample details of gas-liquid embolic agent:

| Sample details | Preparation instructions |
|---|---|
| Carbon dioxide- | Quantity: 2 pcs |
| normal saline | |
| Conduit 1 | Carbon dioxide:alcohol:ioversol = 22:35:15 |
| Conduit 2 | Carbon dioxide:alcohol:ioversol = 22:35:15 |
| Conduit 3 | Carbon dioxide:alcohol:ioversol = 42:35:15 |
| Conduit 4 | Carbon dioxide:alcohol:ioversol = 32:35:15 |
| Conduit 5 | Carbon dioxide:alcohol:ioversol = 39:35:15 |
| Conduit 6 | Carbon dioxide:alcohol:ioversol = 27:35:15 |
| Conduit 7 | Carbon dioxide:alcohol:ioversol = 47:35:15 |
| alcohol | 75% |
| Ioversol | 75% |
| Conduit | Material: Teflon, inner diameter: 2 mm, |
| | outer diameter: 2.4 mm, length: 1,000 mm |

5.3.2 The basic conditions of the left kidney, the right kidney and the liver were observed through B-ultrasound.

5.3.3 Carbon dioxide-normal saline was injected through the left kidney artery, 22 μl of carbon dioxide entered the blood vessel, and the gas was basically absorbed and the blood flowed normally by ultrasonic observing after 2 min.

5.3.4 Pre-embolization angiography was performed on the left kidney artery through the conduit 1; the embolic agent was injected; the injector scales were respectively at 1.7 ml and 1.5 ml in the period from the embolic agent starting to enter the blood vessel to blood flowing back, and it could be calculated that 0.2 ml of the embolic agent entered the target blood vessel, including about 160 μl of 75% alcohol; and ideal gas-liquid clarity was achieved. It was observed that the upper pole of the left kidney was embolized and the lower pole of the left kidney was not embolized, and by preliminary judgment, it was possibly caused by that normal saline-gas in the micro-conduit entered the lower pole of the left kidney in advance along with blood flow, which caused certain obstruction so that alcohol injected later could basically enter the upper pole of the left kidney.

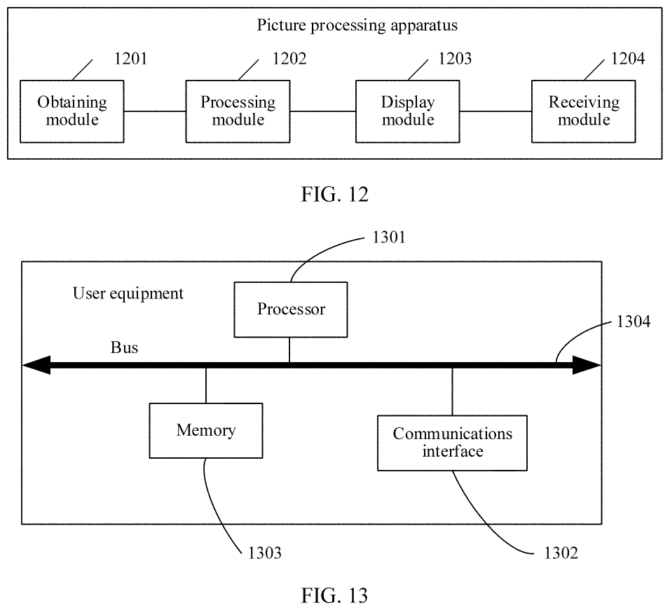

5.3.4.1 The angiography image obtained immediately after embolization of the left kidney was shown in FIG. 15A, and it was shown through ultrasound that blood flow basically disappeared (FIG. 15B).

5.3.4.2 The angiography image obtained 5 min after embolization of the left kidney was shown in FIG. 15C.

5.4 The gas-liquid embolic agent was injected through hepatic artery by using the conduit 2-4, and 4.2 ml of the injected embolic agent was observed when blood flowed back, including about 1,100 μl of 75% alcohol.

5.4.1 Pre-embolization angiography of liver was shown in FIG. 15D.

5.4.2 Angiography performed 5 min after liver embolization was shown in FIG. 15E.

5.5 The gas-liquid embolic agent was injected through carotid artery by a conduit 5, and the injected gas-liquid embolic agent contained about 400 μl of 75% alcohol and about 600 μl of carbon dioxide. DSA observation showed that part of alcohol and carbon dioxide entered distal internal jugular vein.

5.5.1 Pre-embolization angiography of internal jugular artery was shown in FIG. 15F.

5.5.2 Immediate angiography after internal jugular artery embolization was shown in FIG. 15G, showing that internal jugular artery was completely embolized.

It was emphasized that in the above description, all embodiments and all experimental data were distinguished only for clearer description. Those of ordinary skill in the art could understand that the formulas of the fillers in the conduits in all the embodiments, the conduits of various specifications, the mode of injecting liquid or gaseous fillers with the normal-temperature solubility lower than 1% into the conduits on a surgery site or the mode of utilizing the stored and transported conduits (the conduits were pre-filled with the liquid or gaseous fillers with the solubility lower than 1%) and the like could be combined to form novel technical solutions, and it was not limited to the combination solutions in the above embodiments.

The pharmaceutical preparation with the tracing function and the delivery system therefor were described in detail above. For a person of ordinary skill in the art, any obvious modifications made to the present disclosure without departing from the essence of the present disclosure will constitute an infringement of patent rights of the present disclosure, and corresponding legal liabilities will be born.

1. A pharmaceutical preparation with a tracing function, comprising a conduit for containing a tracer drug, and a conduit head, wherein
a first drug and a second drug in a liquid or gaseous state are arranged in the conduit, and the first drug and the second drug are each divided into multiple sections, which are arranged in series at intervals in the conduit;
one of the first drug and the second drug is the tracer drug that can be developed in a medical imaging device in the human body; and
the first drug and the second drug are immiscible, insoluble or slightly soluble, and satisfy the acceptable treatment compatibility requirements in the art.

2. The pharmaceutical preparation according to claim 1, wherein
the first drug is a contrast agent, and the second drug is a gaseous separant.

3. The pharmaceutical preparation according to claim 1, wherein
the first drugs in the liquid state or the second drugs in the liquid state are at two ends of the conduit.

4. The pharmaceutical preparation according to claim 1, wherein
multiple sections of third drug in a gaseous or liquid state are also arranged in the conduit, and
the third drug is arranged between the first drug and the second drug; and
the third drug is immiscible with the first drug and the second drug and satisfies the acceptable compatibility requirements in the art; and the third drug and the second drug satisfy the compatibility requirements.

5. The pharmaceutical preparation according to claim 4, wherein
the first drug which is the tracer drug is a liquid contrast agent and positioned at the two ends of the conduit;
the second drug is a gaseous separant, and the first drug is positioned on the two sides of each section of the second drug;
the third drug is an embolic agent or a perfusion agent, and the second drug is positioned on the two sides of each section of the third drug; and
arranging the first drug, the third drug, the second drug and the third drug in the conduit from the end is served as a unit and is repeatedly arranged until the first drug is positioned at the other end of the conduit.

6. The pharmaceutical preparation according to claim 5, wherein
the first drug is an anhydrous iodine contrast agent, the second drug is carbon dioxide, and the third drug is alcohol.

7. An aerobic contrast agent, comprising a conduit and a conduit head, wherein oxygen and the liquid contrast agent are arranged in the conduit, and the oxygen and the contrast agent are each divided into multiple sections which are arranged in series at intervals in the conduit; and
the oxygen and the liquid contrast agent are immiscible, insoluble or slightly soluble, and satisfy the acceptable treatment compatibility requirements in the art.

8. An aerobic embolic agent, comprising a conduit and a conduit head, wherein oxygen, a liquid contrast agent and the liquid embolic agent are arranged in the conduit; the oxygen, the contrast agent and the embolic agent are each divided into multiple sections; and the contrast agent and the embolic agent are arranged in series at intervals in the conduit through the oxygen; and
the oxygen, the liquid contrast agent and the liquid embolic agent are immiscible, insoluble or slightly soluble, and satisfy the acceptable treatment compatibility requirements in the art.

9. An aerobic perfusion agent, comprising a conduit and a conduit head, wherein oxygen, a liquid contrast agent and the liquid perfusion agent are arranged in the conduit and are each divided into multiple sections; and the contrast agent and the embolic agent are arranged in series at intervals in the conduit through the oxygen; and
the oxygen, the liquid contrast agent and the liquid perfusion agent are immiscible, insoluble or slightly soluble, and satisfy the acceptable treatment compatibility requirements in the art.

10. A delivery system for a pharmaceutical preparation with a tracing function, comprising an injection pump, the conduit according to claim 1, a sheath tube holder and a puncture needle which are sequentially connected.

11. The delivery system according to claim 10, wherein
the conduit is connected with the injection pump and the sheath tube holder through Luer tapers.

12. The delivery system according to claim 10, wherein
the inner diameter of the conduit is equal to or larger than that of a conduit sheath.
